(19) United States
(12) Patent Application Publication
(10) Pub. No.: US 2013/0291135 A1
(43) Pub. Date: Oct. 31, 2013

(54) TRANSGENIC MODEL OF ALZHEIMER'S DISEASE

(71) Applicants: Jun Tan, Tampa, FL (US); Demian Forest Obregon, Tampa, FL (US); Huayan Hou, Tampa, FL (US)

(72) Inventors: Jun Tan, Tampa, FL (US); Demian Forest Obregon, Tampa, FL (US); Huayan Hou, Tampa, FL (US)

(73) Assignee: UNIVERSITY OF SOUTH FLORIDA, Tampa, FL (US)

(21) Appl. No.: 13/926,610

(22) Filed: Jun. 25, 2013

Related U.S. Application Data
(63) Continuation of application No. PCT/US2012/022549, filed on Jan. 25, 2012.
(60) Provisional application No. 61/436,040, filed on Jan. 25, 2011.

(51) Int. Cl. A01K 67/027

(52) U.S. Cl. CPC A01K 67/0275
USPC 800/12; 800/22

(57) ABSTRACT

Evidence indicates dysregulation. of the immunoregulatory molecule CD45 occurs in Alzheimer's disease (AD). Transgenic mice overproducing amyloid-β peptide (Aβ) and deficient in CD45 (PSAPP/CD45⁻/⁻) recapitulate AD neuropathology. Increased cerebral intracellular and extracellular soluble oligomeric and insoluble Aβ, decreased plasma soluble Aβ increased microglial neurotoxic cytokines TNF-α and IL-1β, and neuronal loss were found in PSAPP/CD45⁻/⁻ mice compared with CD45-sufficient PSAPP littermates. After CD45 ablation, in vitro and in vivo studies demonstrate a microglial phenotype whereby microglia phagocytose less Aβ but display proinflammatory properties. This microglial activation occurs with elevated Aβ oligomers and neural injury and loss as determined by decreased ratio of anti-apoptotic Bcl-xL to proapoptotic Bax, increased activated caspase-3, mitochondrial dysfunction, and loss of cortical neurons in PSAPP/CD45⁻/⁻ mice. These data show that deficiency in CD45 activity leads to brain accumulation of neurotoxic Aβ oligomers and validate CD45-mediated microglial clearance of oligomeric Aβ as a novel AD therapeutic target.

### CROSS REFERENCE TO RELATED APPLICATIONS

This application is a continuation of prior filed International Application, Serial Number PCT/US2012/22549 filed Jan. 25, 2012, which claims priority to U.S. Provisional Patent Application No. 61/436,040, entitled “Method of Treating Alzheimer's Disease”, filed on Jan. 25,2011, the contents of which are herein incorporated by reference.

### STATEMENT OF GOVERNMENTAL SUPPORT

This invention was made with Government support under Grant Nos. AG04418/Project-2 and R01AG032432 awarded by the National Institutes of Health (NIH)/National Institute on Aging (NIH) and Grant No. R01NS048335 awarded by the National Institutes of Health (NIH)/National Institute of Neurological Disorders and Stroke. The Government has certain rights in the invention

### FIELD OF INVENTION

This invention relates to Alzheimer's disease models. Specifically, the invention provides a transgenic model of Alzheimer's disease.

### BACKGROUND OF THE INVENTION

Converging lines of evidence, indicate dysregulation of the key immauoregulatory molecule CD45 (also known as leukocyte common antigen) in Alzheimer's disease (AD). Transgenic mice that overproduced amyloid-β peptide (Aβ) but were deficient in CD45 (PSAPP/CD45⁻/⁻ mice) were found to faithfully recapitulate AD neuropathology. Specifically, increased abundance of cerebral intracellular and extracellular soluble oligomeric and insoluble Aβ, decreased plasma soluble Aβ, increased abundance of microglial neurotoxic cytokines tumor necrosis factor-α and interleukin-1β, and neuronal loss in PSAPP/CD45⁻/⁻ mice compared with CD45-sufficient PSAPP littermates (bearing mutant human amyloid precursor protein and mutant human presenilin-1 transgenes). After CD45 ablation, in vitro and in vivo studies demonstrate a microglial phenotype in which microglia phagocytose less Aβ phagocytic but display proinflammatory properties. This form of microglial, activation occurs with elevated Aβ oligomers and neural injury and loss as determined by decreased ratio of anti-apoptotic Bcl-xL to proapoptotic Bax, increased activated caspase-3, mitochondrial dysfunction, and loss of cortical neurons in PSAPP/CD45⁻/⁻ mice. These data show that deficiency in CD45 activity leads to brain accumulation of neurotoxic Aβ oligomers and validate CD45-mediated microglial clearance of oligomeric Aβ as a novel AD therapeutic target.

Deposition of amyloid-β peptide (Aβ) as β-amyloid plaques is a defining pathological hallmark of Alzheimer's disease (AD) and occurs with increased abundance of soluble Aβ and activation of microglia-mediated inflammatory responses (Sedgwick, et al., (1991) Isolation and direct characterization of resident microglial cells from the normal and inflamed central nervous system. Proc Natl Acad Sci USA 88:7438 -7442). However, reactive microglia ultimately fail to clear Aβ in brains of AD patients and in mouse models of the disease (McGeer, et al,, (1987), Reactive microglia in patients with senile dementia of the Alzheimer type are positive for the histocompatibility glycoprotein HLA-DR (Neurosci Lett 79:195-200; Benzing, et al, 1999, Evidence for glial-mediated inflammation in aged APP(SW) transgenic mice (Neurobiol Aging 20:581-589). It has even been suggested that chronic microglial immune responses contribute to AD pathogenesis by promoting Aβ plaque formation (Frackowiak, et al., 1992). Ultrastructure of the microglia that phagocytose amyloid and the microglia that produce beta-amyloid fibrils (Acta Neuropathol. 84:225-233; Wisniewski and Frackowiak, 1998) Commentary to “Differences between the pathogenesis of senile plaques and congophilic angiopathy in Alzheimer disease” (J Neuropathol Exp Neurol, 1997) 56:751-761; J Neuropathol Exp Neurol 57:96-98; Townsend, et al, 2005) CD40 signaling regulates innate and adaptive activation of microglia in response to amyloid beta-peptide (Ear J Immunol 35:901-910), but the molecular mechanisms underlying this deleterious response have rammed elusive.

CD45 (also known as leukocyte common antigen), the most abundant transmembrane protein tyrosine phosphatase, is expressed on all nucleated hematopoietic cells and plays an important role in regulating immune responses (Thomas and Brown, 1999) Positive and negative regulation of Src family membrane kinases by CD45 (Immunol Today 20:406-411: Penninger, et al., 2001; CD45: new jobs for an old acquaintance, Nat Immunol 2:389-396). In the periphery, CD45 promotes antigen-specific B- and T-cell responses by dephosphorylating Src-family kinases (Thomas and Brown, 1999) Positive and negative regulation of Src-family membrane kinases by CD45 (Immunol Today 20:406-411; Penninger. et al,, 2001; CD45: new jobs for an old acquaintance. Nat Immunol 2:389-396). CD45 plays additional roles in regulating selectin expression (Stibenz, et al., 1996; CD45 engagement induces L-selectin down-regulation. Scand J Immunol 44:37-44; Wrobiewski and Hamann, 1997 CD45-mediated signals can trigger sheddingof lymphocyte L-selectin. lot Immunol 9:555-562) and integral function (Roach, et al,, 1997) CD45 regulates Src family member kinase activity associated with macrophage integrin-mediated adhesion. Curr Biol 7:408-417; Shenoi, et al,, (1999) Regulation of integrin-mediated T cell adhesion by the transmembrane protein tyrosine phosphatase CD45 (J Immunol 162:7120-7127). CD45 has also been shown to negatively regulate cytokine receptor mediated signaling via Janus associated kinases (Irie-Sasaki, et al., 2001) CD45 is a JAK phosphatase and negatively regulates cytokine receptor signalling (Nature 409:349-354), revealing yet another role of CD45 in dampening overly exuberant immune responses,

Resting microglia constitutively express CD45 in vitro, which is further inducible at the cell surface during activation (Sedgwick, et al., (1991) Isolation and direct characterization of resident microglial cells from the normal and inflamed central nervous system (Proc Natl Acad Sci USA 88:7438 -7442; Carson, et al., 1998) Mature microglia, resemble immature antigen-presenting cells (Glia 22:72-85). Importantly, microglia in the frontal cortex and hippocampus of normal aging individuals express CD45, and expression abundance is markedly increased in close vicinity of β-amyloid plaques in AD patient brains (Masliah, et al., 1991), Immunoreactivity of CD45, a protein phosphotyrosme phosphatase, in Alzheimer's disease (Acta Neuropathol 83:12-20) and in transgenic .mouse models of the disease (Wilcock, et al, 2001) Number of Abeta inoculations in APP+PSl transgenic mice influences antibody titers, microglial activation, and congophilic plaque levels (DNA Cell Biol 20:731-736; Maier, et al., 2008) Complement C3 deficiency leads to accelerated amyloid beta plaque deposition and neurodegeneration and modulation of the microglia/macrophage phenotype in amyloid precursor protein transgenic mice (J Neuroses 28:6333-6341). Stimulation of microglial CD45 opposes CD40 ligand (CD40L)-induced activation of the Src-family kinases Lek and Lyn, which are key transducers of proinflammatory innate immune responses (Tan, et al., 2000), CD45 inhibits CD40L-induced microglial activation via negative regulation of the Src/p44/42 MAPK pathway (J Biol Chem 275:37224-37231). Co-treatment of microglia with CD40L and agonistic CD45 antibody abrogates microglial tumor necrosis factor-□ (TNF-□) production via inhibiting p44/42 mitogen-activated protein kinase (MAPK) activity; a downstream signaling event resulting from Src-family kinase activation (Tan. et al., 2000). CD45 inhibits CD40L-induced microglial activation via negative regulation of the Src/p44/42 MAPK pathway (J Biol Chem 275:37224-37231; Tan, et al., 2000). CD45 opposes p-amyloid peptide-induced microglial activation via inhibition of p44/42 mitogen-activated protein kinase (J Neurosci 20:7587-7594). Thus, stimulation of the CD45 signaling pathway suppresses proinflammatory microgliosis that is etiologically implicated in neurodegenerative disorders, including AD (Akiyaraa, et al, 2000). Inflammation and Alzheimer's disease (Neurobiol Aging 21:383- 421; Tan, et al., 2000). CD45 opposes β-amyloid peptide-induced microglial activation via inhibition of p44/42 mitogen-activated protein kinase (J Neurosci 20:7587-7594; Penninger, et at, 2001: CD45: new jobs for an old acquaintance, Nat Immunol 2:389-396).

While the use of isolated cell lines is helpful in elucidating the role of proteins on immune system regulation in amyloid diseases, more holisitie data is only obtained from observation directly in a live mammal, i.e. an in vivo model. As such, mammal models have been generated with altered levels of gene expression or expression of non-endogenous from other species including human genes. For example, transgenic models have a novel gene or genes introduced into the animal genome, such as those described by Leder, et al. (U.S. Pat. No. 4,736,866, issued Apr. 12, 1988), and Krimpenfor, et al. (U.S. Pat. No. 5,175,384, issued Dec. 29, 1992), and Terhorst, et al. (WO/1992/Q22645published Dec. 23, 1992). Preparation of a knockout mammal requires introducing nucleic acid constructs that suppress expression of a particular gene into an embryonic stem cell, which is then introduced into an embryo for incorporation.

However, there are no live models which reconstruct well the amyloid diseases. As such, what is needed is an organism which provides a more accurate model of amyloid diseases.

### SUMMARY OF THE INVENTION

To elucidate the role of CD45 in AD-like pathology, a genetic approach to cross doubly transgenic PSAPP mice was used. PSAPP mice develop accelerated cerebral amyloidosis. A mouse model of amyloid disease was prepared which has a haplotype derived from a PSAPP mouse and a haplotype derived from a CD45 deficient mouse. The PSAPP haplotype is optionally derived from a double transgenic “Swedish” APPK595N/M596L strain, mouse and a PS1E9 B6C3-Tg 85Dbo/J strain mouse. Likewise, the CD45 deficient haplotype is optionally derived from a B6.129-Ptprctml Holm/J strain mouse. The mouse model thus exhibits an elevated level of amyloid proteins and impaired amyloid clearance. The elevated levels of amyloid proteins are compared to wild-type mice, and wherein the amyloid proteins are dimeric Aβ, oligomeric Aβ, or a combination thereof. To reduce sex differences on Aβ deposition, in some variations, only female mice are compared.

In some variations of the moose model wherein model shows elevated levels of total soluble intracellular Aβ species. These elevated levels may be detergent-soluble Aβ however in the CNS the clearance of these species from the CHS is likely decreased thus resulting in toxic accumulation of A□ species. Some models also exhibit mitochondrial dysfunction.

Also disclosed is a method of forming the mouse model of amyloid disease by obtaining a first filial parent having a genotype derived from a PSAPP mouse, and a second filial parent having a genotype derived from a CD45 deficient mouse. Optionally, the first filial parent overproduces Aβ. The two filial parents are then interbred to form first generational mouse model having a heterozygous PSAPP haplotype and a homozygous CD45-deficient haplotype. The PSAPP mice were maintained as heterozygotes by crossing transgenic mice to wild-type B6C3F1/J mice. The mouse model is screened for PSAPP and CD45 genotypes. In some variations, the screening is performed by PCR from genomic DMA.

### BRIEF DESCRIPTION OF THE DRAWINGS

For a fuller understanding of the invention, reference should be made to the following detailed description, taken in. connection with the accompanying drawings, in which:

FIGS. 1(A) through (D) are images depicting accelerated cerebral amyloidosis in PSAPP/D45⁻/⁻ mice. Mouse brain sections from (A, B) PSAPP/CD45 and (C, D) PSAPP/CD45−/− mice stained with (A, C) Aβ OC antibody (dark gray) and DAPI (light gray) at 4 months of age or (B, D) ThioS at 8 months of age.

FIGS. 2(A) and (B) are images showing age-dependent increases in cerebral Aβ in PSAPP/CD45^{-/-} mice. Mouse brain sections from eight-month-old (A) PSAPP/CD45 and (B) PSAPP/CD45^{-/-} mice were stained with Aβ oligomer/conformational (OC) antibody (dark gray) and DAPI (light gray).

FIG. 3 is a graph showing age-dependent increases in cerebral Aβ in PSAPP/CD45^{-/-} mice. Aβ burden (including cingulate cortex, hippocampus, and entorhinal cortex) was calculated for OC iramtraoreactivity by quantitative image analysis. Data are represented as mean±SD with n=8 females/group at eight months of age.

FIG. 4 is a blot showing age-dependent increases in cerebral Aβ in PSAPP/CD45^{-/-} mice. Brain bomogenates were prepared from four-month-aid PSAPP/CD45, PSAPP/CD45^{-/-}, and wild-type mice. Western blot by antibody 6E10 shows increased abundance of oligomeric Aβ species in brain homogenates from PSAPP/CD45^{-/-} vs. PSAPP/CD45 or wild-type (WT) mice.

FIG. 5 is a blot showing brain homogenates prepared from 8-month-old wild-type (WT), PSAPP/CD45, or PSAPP/CD45−/− mice. Western blot by antibody 6E10 shows increased abundance of dimeric and oligomeric Aβ species in brain homogenates from PSAPP/CD45−/− versus PSAPP/CD45 or wild-type mice.

FIGS. 6(A) and (B) are graphs depicting simultaneously increased central and reduced peripheral. Aβ in PSAPP/CD45−/− mice. Mouse brain homogenates were prepared from PSAPP/CD45−/− and PSAPP/CD45 mice at 4 months of age, (A) Detergent-insoluble (5 M guanidine-soluble) total Aβ species (including Aβ_{1-40, 42}; picograms per milligrams of protein) were biochemically assessed in brain homogenates by ELISA. Data are presented as mean±SD (n=8 females per group). (B) The steady-state cerebral and plasma total, soluble Aβ species (including Aβ_{1-40, 42}) at the time of death by ELISA. Aβ levels are represented as percentages versus PSAPP/CD45 mice with mean±SD (n=8 females per group). **p<0.01.

FIGS. 7(A) and (B) are graphs showing PSAPP/CD45^{-/-} mice have simultaneously altered cerebral and peripheral soluble Aβ species. Mouse brain, homogenates were prepared from PSAPP/CD45^{-/-} and PSAPP/CD45 mice at eight months of age. (A) Detergent-insoluble (5 M guanidine-soluble) total Aβ species (including Aβ_{1-40, 42}; pg/mg of protein) were biochemically assessed by ELISA. Data are represented as mean±SD (n=8 females/group). Cerebral total insoluble Aβ species did not differ between PSAPP/CD45^{-/-} and PSAPP/CD45 mice at eight months of age. (B) Steady-state cerebral and plasma total soluble Aβ species (including Aβ_{1-40, 42}) were analyzed at sacrifice by ELISA. Aβ levels are represented as percentages over PSAPP/CD45 mice with mean±SD (n=8 females/group).

FIG. 8 is a graph showing PSAPP/CD45^{-/-} mice have a pro-inflammatory microglial phenotype. CD11b or CD40 green fluorescence area in entorhinal cortex was calculated by quantitative image analysis, and similar results were obtained in cingulate cortex and hippocampus (data not shown). Data are represented as mean±SD with n=8 females/group at eight months of age.

FIGS. 9(A) and (B) are graphs showing the inflammatory microglial phenotype of PSAPP/CD45−/− mice. The microglial proinflammatory cytokines TNF-α and IL-1β were quantified in brain homogenates. from both PSAPP/CD45 and PSAPP/CD45−/− mice at (A) 4 and (B) 8 months of age by ELISA. Data are represented as mean±SD (n=8 female mice per group) for each cytokine (picograms per milligrams of protein). **p<0.01.

FIGS. 10(A) and (B) are graphs showing CD45-deficient primary microglia have impaired Aβ₁₋₄₂ phagocytosis. CD45-sufficient or -deficient primary microglial cells were prepared from neonatal mice and treated with agonistic CD45 antibody (2.5 μg/ml) or isotype control IgG (data not shown) in the presence of 1 μM aged FITC-Aβ₁₋₄₂ for 60 min. Cellular supernatants and lysates were analyzed for (A) cell-associated and (B) extracellular FITC-Aβ₁₋₄₂ using a fluorometer. Data are represented as the relative fold of mean, fluorescence change (mean±SD), calculated as the mean fluorescence for each sample at 37° C. divided by mean fluorescence at 4° C. (n=6 for each condition presented). *p<0.05, **p<0.01.

FIG. 11(A) through (F) are images showing intraneuronal Aβ accumulates in PSAPP/CD45^{'/-} mice. Mouse brain sections from (A-C) four-month-old PSAPP/CD45 and (D-F) four-month-old PSAPP/CD45^{-/-} mice were reacted with Aβ antibody 6E10, and signals were primarily within neurons in cortical regions and in hippocampus (entorhinal cortex is shown). Magnification is (A, D) 10×; (B,E) 20×; and (C,F) 40×.

FIG. 12(A) through (F) are images showing intraneuronal Aβ accumulates in PSAPP/CD45^{-/-} mice. Mouse brain sections from (A-C) eight-month-old PSAPP/CD45 and (D-F) four-month-old PSAPP/CD45^{-/-} mice were reacted with Aβ antibody 6E10, and signals were primarily within neurons in cortical regions and in hippocampus (entorhinal cortex is shown). Magnification is (A, D) 10×; (B,E) 20×; and (C, F) 40×.

FIGS. 13(A) and (B) are graphs showing increased intracellular Aβ in PSAPP/CD45−/− mice. (A) Extracellular and (B) intracellular proteins were prepared from 8-month-old wild-type (WT), PSAPP/CD45, and PSAPP/CD45−/− mouse brain homogenates. Western blot analysis by antibody 6E10 shows increased abundance of Aβ d inters and oligomers in brain extracts from PSAPP/CD45−/−versus PSAPP/CD45 or wild-type mice. Results are represented as means±SD (n=8 females per group) of total soluble Aβ species (picograms per milligrams of protein). *p<0.05, **p<0.01.

FIG. 14 is a blot showing intraneuronal Aβ accumulates in PSAPP/CD45^{-/-} mice. Extracellular proteins were prepared from four-month-old wild-type, PSAPP/CD45 and PSAPP/CD45^{-/-} mouse brain extracts. Western blot analysis by antibody 6E10 shows increased abundance of Aβ oligomers in brain extracts from PSAPP/CD45^{-/-} vs. PSAPP/CD45 or wild-type mice at 4 months of age.

FIG. 15 is a blot showing intraneuronal Aβ accumulates in PSAPP/CD45^{-/-} mice. Intracellular proteins were prepared from four-month-old wild-type, PSAPP/CD45 and PSAPP/CD45^{-/-} mouse brain extracts. Western blot analysis by antibody 6E10 shows increased abundance of AP oligomers in brain extracts from PSAPP/CD45^{-/-} vs. PSAPP/CD45 or wild-type mice at 4 months of age.

FIGS. 16(A) and (B) are graphs showing increased intracellular Aβ in PSAPP/CD45−/− mice. Total detergent-soluble Aβ species (including Aβ_{1-40, 42}) in (A) extracellular or (B) intracellular fractions were assayed in 4- and 8-month-old PSAPP/CD45 and PSAPP/CD45−/− mouse brain extracts by ELISA. Results are represented as mean±SD (n=8 females per group) of total soluble Aβ species (picograms per milligrams of protein). *p<0.05, **p<0.01.

FIGS. 17(A) through (I) are images showing neuronal injury and loss. PSAPP/CD45−/− mice have neuronal injury and loss. Moose brain sections from (A-C) 8-month-old CD45−/−, (D-F) 8-month-old PSAPP/CD45, and (G-I) 8-month-old PSAPP/CD45−/− mice were stained with Nissl (dysmorphic neurons are indicated by arrows). *p<0.05. Magnification is (A, D, G) 10×; (B,E, H) 20×; and (C, F, I) 40×.

FIGS. 18(A) through (I) are images showing neuronal injury and loss. PSAPP/CD45−/− mice have neuronal injury and loss. Representative entorhinal cortex brain sections from (A-C) 8-month-old CD45−/−, (D-F) 8-month-old PSAPP/CD45, and (G-I) 8-month-old PSAPP/CD45−/− mice were stained with NeuN, *p<0.05. Magnification is (A, D, G) 4×; (B,E, H) 10×; and (C, F, I) 20×.

FIG. 19 is a graph showing neuronal injury and loss. Stereological analysis for NeuN-positive cells in the medial entorhinal cortex (MEA) (n_{—}6 female mice per group; mean±SD) is graphically represented. *p<0.05.

FIG. 20 is a blot showing neuronal injury and loss. Brain homogenates were prepared from 8-month-old control CD45−/−, PSAPP/CD45, and PSAPP/CD45−/− mice and probed by Western, blot using antibodies against NeuN, Bcl-xL, or Bax. Note reduced expression of NeuN and Bcl-xL and increased abundance of Bax protein in PSAPP/CD45−/−versus PSAPP/CD45 or CD45−/−mouse brains. WT, Wild type. *p<0.05.

FIG. 21 is a blot showing neuronal injury and loss. Brain homogenates were prepared from 8-month-old control CD45−/−, PSAPP/CD45, and PSAPP/CD45−/− mice and probed by Western blot using antibodies against total and cleaved (active) caspase-3. Note increased abundance of cleaved caspase-3 in PSAPP/CD45−/−versus PSAPP/CD45 or CD45−/−mouse brains, WT, Wild type. *p<0.05.

FIGS. 22(A) and (B) are graphs showing dysfunctional mitochondrial in PSAPP/CD45^{-/-} mice. Mitochondria were isolated from (A) cortical, regions (including frontal, entorhinal, and cingulate areas) and (B) hippocampi of eight month-old wild-type, CD45^{-/-}, PSAPP/CD45 or PSAPP/CD45^{-/-} mice. PSAPP/CD45^{-/-} mice had reduced basal (state II) respiratory rate compared with wild-type, CD45^{-/-} or PSAPP/CD45 mice.

FIGS. 23(A) and (B) are graphs showing dysfunctional mitochondrial in PSAPP/CD45 mice. Mitochondria were isolated from (A) cortical regions (including frontal entorhinal, and cingulate areas) and (B) hippocampi of eight month-old wild-type, CD45^{-/-}, PSAPP/CD45 or PSAPP/CD45^{-/-} mice, (a) PSAPP/CD45^{-/-} mice had attenuated maximum respiratory rate compared with wild-type, CD45^{-/-} or PSAPP/CD45 mice.

FIGS. 24 (A) and (B) are graphs showing dysfunctional mitochondrial in PSAPP/CD45^{-/-} mice. Mitochondria were isolated from (A) cortical regions (including frontal, entorhinal, and cingulate areas) and (B) hippocampi of eight month-old wild-type, CD45^{-/-}, PSAPP/CD45 or PSAPP/CD45^{-/-} mice. Mitochondrial membrane potential were reduced in PSAPP/CD45^{-/-} vs. wild-type, CD45^{-/-} or PSAPP/CD45 mice.

FIGS. 25 (A) and (B) are graphs showing dysfunctional mitochondrial in PSAPP/CD45^{-/-} mice. Mitochondria were isolated from (A) cortical regions (including frontal, entorhinal, and cingulate areas) and (B) hippocampi of eight month-old wild-type, CD45^{-/-}, PSAPP/CD45 or PSAPP/CD45^{-/-} mice. Reactive oxygen species production were reduced in PSAPP/CD45^{-/-} vs. wild-type, CD45^{-/-} or PSAPP/CD45 mice.

### DETAILED DESCRIPTION OF THE PREFERRED EMBODIMENT

In the following detailed description of the preferred embodiments, reference is made to the accompanying drawings, which form a part hereof, and within, which are shown by way of illustration specific embodiments by which the invention may be practiced. It is to be understood that other embodiments by which the invention may be practiced, or utilized and structural, changes may be made without departing from the scope of the invention.

The invention is directed to a novel mouse model of Alzheimer's disease.

Unless otherwise noted, the terms used herein are to be understood according to conventional usage by those of ordinary skill in the relevant art. In addition to the definitions of terras provided below, definitions of common terms in molecular biology may also be found in Rieger et al., 1991 Glossary of genetics: classical and molecular, 5th Ed., Berlin: Springer- Veriag; and in Current Protocols in Molecular Biology, P. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, tor example, reference to “a cell” can mean that at least one cell can be utilized.

The term “knockout” is to be construed as referring to a partial or complete suppression of the expression of at least one protein.

The terms “CD45” or “CD45R” refer to a cell surface receptor glycoprotein expressed on the surface of hematopoietic cells, such as phagocytic cells. CD45 has multiple isoforms ranging is weight from about 180 Da to about 235 kDa. Different cell lines express different isoforms, and in some cases cells express more than one isoform. As used herein, CD45 refers to ail isoforms.

To elucidate the role of CD45 in AD-like pathology, a genetic approach to cross doubly transgenic PSAPP mice was used [bearing mutant human amyloid precursor protein (APP) and mutant human presenilin-I (PSI) transgenes], which develop accelerated cerebral amyloidosis (Jankowsky, et al, 2001, Co-expression of multiple iransgenes in mouse CNS: a comparison of strategies. Biomol Eng 17:157-165), with, animals deficient in CD45 (Benatar, et al, 1996, Immunoglobulin-mediated signal transduction in B cells from. CD45-deficient mice, J Exp Med 183:329-334), The AD-like pathology of the formed bigenic mice was then examined.

Double transgenic “Swedish” APPK595N/M596L (APPswe)+PS1E9 B6C3-Tg 85Dbo/J strain (PSAPP mice) and CD45-deficient mice (B6.129-PtprctmlHolm/J) (The Jackson Laboratory, Bar Harbor, Me.) were housed and maintained in compliance with protocols approved by the USF institutional Animal Care and Use Committee. PSAPP mice were maintained as heterozygotes by crossing transgenic mice to wild-type B6C3F1/J mice as described in the original report (Jankowsky, et al., 2001, Co-expression of multiple transgenes in mouse CNS: a comparison of strategies, Biomol Eng 17:157-165). First filial offspring were interbred resulting from crossing heterozygous PSAPP mice with homozygous CD45-deficient mice and analyzed four groups of mice at 4 and 8 months of age: nontransgenic/CD45 wild-type (wild-type), PSAPP/CD45 wild-type (PSAPP), nontransgenic/CD45−/− (CD45−/−) PSAPP/CD45−/− offspring. Animals were screened for PSAPP and CD45 genotypes by PCR from genomic DNA. CD45 genotype was further confirmed by flow cytometry. Because sex differences can impact Aβ deposition (Jankowsky, et al., 2001, Co-expression of multiple transgenes in mouse CNS; a comparison of strategies, Biomol Eng 17:157-165), only females were used in some comparisons.

Mice were killed under isoflurane anesthesia, and 0.5 ml of blood was collected from the heart. Plasma was then separated and stored at −80° C. for later analysis of Aβ levels. Animals were then transcardially perfused with ice-cold PBS. Brains were rapidly isolated and the right hemisphere was snap-frozen on dry ice and stored at −80° C. before protein extraction. The left hemisphere was placed in 4% paraformaldehyde (PEA) in 0.1MPBS overnight and then transferred to a graded series of sucrose solutions (10, 20, and 30%, each at 4° C. overnight) for cryoprotection. Sequential 25 or 40 μm frozen coronal sections were cut using a sliding microtome. Free-floating sections were then stored at 4° C. in 24-well plates containing PBS with 100 mM of sodium azide.

Murine primary cultured microglia were isolated from mouse cerebral cortices and grown in complete RPMI 1640 medium according to previously described methods (Zhu, et al., 2008, CD45RB is a novel molecular therapeutic target to inhibit Abeta peptide-induced. microglial MAPK activation, PLoS One 3:e2135). Briefly, cerebral cortices from newborn, mice (1-2 d old) were isolated under sterile conditions and kept at 4° C. before mechanical dissociation. Cells were grown in RPMI 1640 medium supplemented with 5% heat inactivated FCS, 2 mM glutamine, 100 U/ml penicillin, 100 μg/mt streptomycin, and 50 nM 2-mercaptoethanol. Primary cultures were kept for 14 d so that only glial cells remained. Astrocytes were separated from microglial cultures using a mild trypsinization protocol as described (Saura, et al., 2003, High-yield isolation of murine microglia by mild trypsinization, Glia 44:183-189), More than 98% of these glial cells stained positive for Mac-1 (F. Hoffmann-La Roche Ltd., Basel, CH) by flow cytometry.

For specific extraction of extracellular versus intracellular proteins, hemibrains were harvested and placed in 500 μl of solution containing 50 mM Tris-HCl, pH 7.6, 0.01% NP-40, 150 mM NaCl, 2 mM EDTA, 0.1% SDS, 1 mM phenylmerhylsulfonyl fluoride, and protease inhibitor cocktail (Sigma) as described (Lesné, et al., 2006, A specific amyloid-beta protein assembly in the brain impairs memory. Nature 440:352-357). Soluble, extracellular proteins were collected from mechanically homogenized lysates after centriIligation for 5 min at 3000 rpm. Cytoplasmic proteins were extracted from cell pellets mechanically dissociated with a micropipettor in 500 μl of TNT buffer (50 mM Tris-HCl, pH 7.6, 150 mM NaCl, and 0.1% Triton X-100) after centrifugation for 90 min. at 13,000 rpm. Insoluble material was incubated with 20 μl of 70% formic acid, mechanically dissociated with a micropipette, gently agitated for 1 h, and buffered with 380 μl of 1 M Tris-HCl, pH 8.0. Samples were centrifuged for 90 rain at 13,000 rpm, and supernatants were collected for analysis.

For total protein, extraction, brains were removed and hemibrains were snap-frozen on dry ice and stored at −80° C. Samples were subsequently homogenized in immunoprecipitation assay buffer containing the following: 150 mM NaCl, 50 mM Tris, pH 7.4, 0.5% deoxycholic acid, 0.1% SDS, 1% Triton X-100, 2.5 mM EDTA, and protease inhibitor cocktail Protein concentration was measured in the supernatant by BCA Protein Assay (Thermo Fisher Scientific Inc., Rockford, Ill.).

All data were normally distributed; therefore, in instances of single mean comparisons, Levene's test for equality of the variance followed by/test for independent samples was used to assess significance. In instances of multiple mean comparisons, ANOVA was used, followed, by post hoc comparison using Bonferroni's method. α levels were set at 0.05 for all analyses. The Statistical Package for the Social Sciences release 10.0.5 (SPSS) was used for all data analysis.

### EXAMPLE 1

The cerebral amyloidosis of aged PSAPP mice deficient in CD45 was examined. Brain Aβ deposition is a pathognomonic feature of AD (Selkoe, (2001) Alzheimer's disease: genes, proteins, and therapy. Physiol Rev 81:741-766), and oligomeric Aβ species are thought to be a driving force in AD-type neurodegeneration (Klyubin, et al., (2005) Amyloid beta protein immunotherapy neutralizes Abeta oligomers that disrupt synaptic plasticity in vivo. Nat Med 11:556-561; Walsh., et al., (2005) The role of cell-derived oligomers of Abeta in Alzheimer's disease and avenues for therapeutic intervention. Biochem Soc Trans. 33:1087-1.090; Shankar, et at, (2008) Amyloid-beta protein dimers isolated directly from Alzheimer's brains impair synaptic plasticity and memory. Nat Med 14:837- 842), The double-transgenic PSAPP mouse (Jankowsky, et at, (2001) Co-expression, of multiple transgenes in mouse CNS: a comparison of strategies. Biomol Eng 17:157-165) is a widely used model of cerebral amyloidosis, thus PSAPP mice were bred with animals deficient in CD45 and offspring sacrificed at either 4 or 8 months of age to evaluate changes in AD-like pathology.

Aβ deposits were quantified, by immunofluorescence using six 25 μm free-floating sections spaced 200 μm apart through each anatomic region of interest: (hippocampus and cerebral cortex) as described previously (Tan, et at. (2002) Role of CD40 ligand in amyloidosis In transgenic Alzheimer's mice. Nat Neurosci 5:1288 -1293; Town, et at, (2008) Blocking TGF-beta-Smad2/3 innate immune signaling mitigates Alzheimer-like pathology. Nat Med 14:681-687). Brain sections were immunostained with rabbit polyclonal oligomer/conformational (OC) Aβ antibody (Kayed, et al., (2007) Fibril specific, conformation dependent antibodies recognize a generic epitope common to amyloid fibrils and fibrillar oligomers that is absent in prefibrillar oligomers. Mol Neurodegener 2:18), using Alexa Fluor 488-conjugated goat anti-rabbit secondary antibody (Life Technologies Corporation, Carlsbad, Calif.). Amyloid burden was determined at 20× magnification, by quantitative image analysis using an automated Zeiss Observer Zl inverted microscope with an attached Axiocam MRm CCD camera and Axiovision software version 4.6 (Carl Zeiss A G, Oberkochen, Germany). Quantitative image analysis was performed by a single examiner blinded to sample identities. Data are reported as percentage of positive pixels divided by total pixels captured for each region of interest.

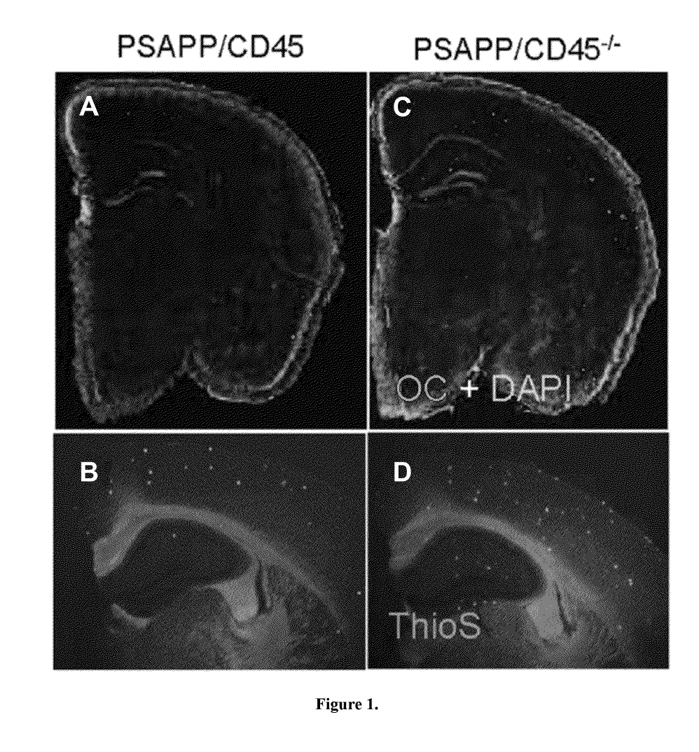

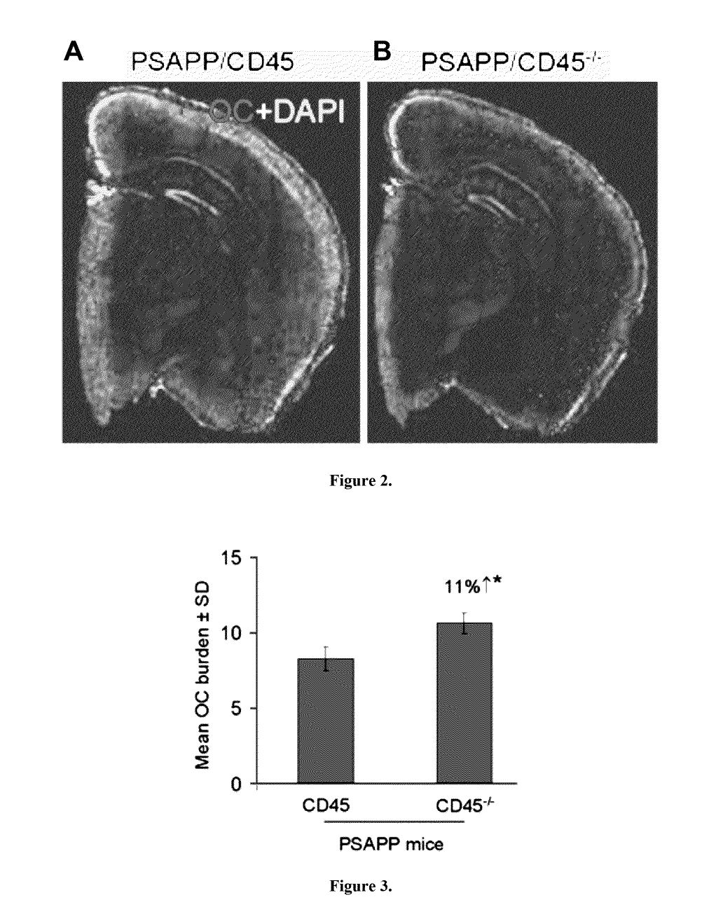

Mouse brain sections were reacted, with OC antibody directed against oligomeric/conformational Aβ species (Kayed, et al., (2007) Fibril specific, conformation dependent antibodies recognize a generic epitope common to amyloid fibrils and fibrillar oligomers that is absent in prefibrillar oligomers. Mol Neurodegener 2:18; Glabe, (2008) Structural classification of toxic amyloid, oligomers. J Biol Chem 283;29639-29643) and counterstained cell nuclei with DAPI, as seen in FIGS. 1(A) and (C) and 2(A) and (B).

Sections were also stained for fibrillar Aβ with ThioS, seen in FIGS. 1(C) and (D). Brain sections were incubated for 5 min. in a 1% thioflavin S (ThioS) (Sigma) solution dissolved in distilled water containing 70% ethanol Tissue sections were then rinsed twice with distilled water and mounted with fluorescence mounting medium containing 4′,6′-diamidino-2-phenylindole (DAPI) (Vector Laboratories, Inc., Burlingame, Calif.), Aβ burden was calculated, for OC or ThioS stains (within cingulate cortex, hippocampus, and entorhinal cortex) by quantitative image analysis, and data represented as mean±SD with n=8 females per group at 4 or 8 months of age. Quantitative image analysis revealed significantly (**p<0.01) increased OC reactivity at 4 months of age and ThioS burden at 8 months of age by 56-60% when comparing PSAPP/CD45−/− to PSAPP/CD45 littermates. By 8 months of age, PSAPP/CB45−/− animals had only modest elevation in OC immunoreactivity, as seen in FIG. 3, suggesting that CD45 deficiency accelerates cerebral amyloidosis as opposed to having a cumulative effect on Aβ pathology. As expected, altered cerebral Aβ abundance in PSAPP/CD45−/− versus PSAPP/CD45 mice was not attributable to differences in APP transgene expression or Aβ metabolism (data not shown).

Aβ peptides are metastahle and can exist as monomelic, dimeric, and higher-molecular-weight oligomeric forms both in vitro and in vivo (Selkoe, (2001) Alzheimer's disease: genes, proteins, and therapy. Physiol Rev 81:741-766; Klyubin, et al, (2005) Amyloid beta protein immunotherapy neutralizes Abeta oligomers that disrupt synaptic plasticity in vivo. Nat Med. 11:556-561; Walsh, et al. (2005) The role of cell-derived oligomers of Abeta in Alzheimer's disease and avenues for therapeutic intervention. Biochem Soc Trans 33:1087-1090; Shankar, et al., (2008) Amyloid-beta protein dinners isolated directly from Alzheimer's brains impair synaptic plasticity and memory. Nat Med 14:837-842). it is becoming clear that Aβ dimers and oligomers are likely the neurotoxic species, because direct m vivo administration of these Aβ conformers injures neurons (Shankar, et al., (2008) Amyloid-beta protein dimers isolated directly from Alzheimer's brains impair synaptic, plasticity and memory. Nat Med 14:837-842). To determine whether CD45 deficiency impacted the metastable equilibrium of Aβ, brain homogenates from PSAPP/CD45 or PSAPP/CD45−/− mice at 4 and 8 months of age were probed by Western immunoblot.

Following the sample preparation as described above, an aliquot corresponding, to 40 μg of total protein was electrophoretically separated using 10% Tris-SDS gels or 10-20% Tris-tricine gels (Bio-Rad Laboratories, Hercules, Calif.) and transferred to polyvinylidene fluoride membranes (Bio-Rad laboratories). As a positive control, Aβ oligomers were prepared from synthetic human Aβ₁₋₄₂ according to published methods (Walsh, et al, (2000) The oligomerization of amyloid, beta-protein begins intracellularly in cells derived from human brain. Biochemistry 39:1083-10839: Lesné, et al., (2006) A specific amyloid-beta protein assembly in the brain impairs memory. Nature 440:352-357). Membranes were blocked for I h at room temperature in Tris buffered saline (TBS) (containing 0.1% Tween 20 with 5% nonfat dry milk) and were then incubated with primary antibodies against mouse monoclonal p -actin (1:4000; Sigma-Aldrich Co. LLC, St. Louis, Mo.). Afterward, membranes were immunoblotted with anti-mouse (1:2000; Cell Signaling Technology, Inc.) IgG secondary antibody conjugated with horseradish peroxidase. Proteins were detected with Super Signal West Femto Maximum. Sensitivity Substrate (Pierce) and BIOMAX-MR Film (Thermo Fisher Scientific).

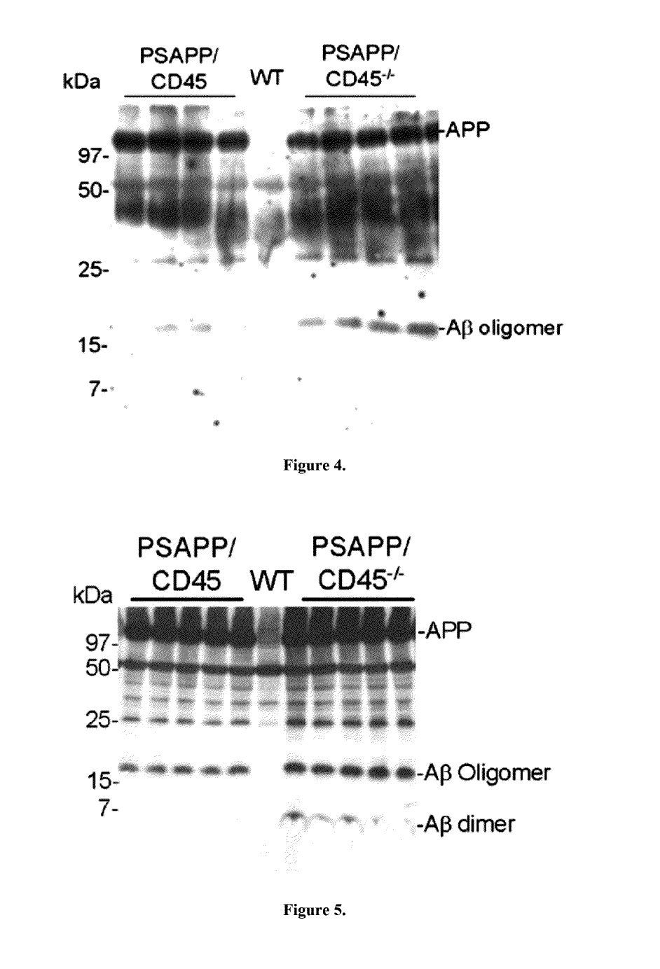

Aβ oligomers were increased in PSAPP/CB45−/− mice at 4 months of age, seen in FIG. 4. Strikingly, both dimeric and oligomeric Aβ species were elevated in PSAPP/CD45−/− versus PSAPP/CD45 mice at 8 months of age, as seen in FIG. 5, Together, these results indicate that cerebral Aβ pathology is overrepresented in CD45-deficient PSAPP mice.

### EXAMPLE 2

The brain-to-blood Aβ clearance capacity of aged PSAPP/CD45−/− mice was examined. It has been proposed that cerebral Aβ is cleared across the blood-brain barrier (BBB) via a “peripheral sink,” and there is evidence of dysfunctional brain-to-blood Aβ clearance in AD patients and in transgenic mouse models of the disease (DeMattos, et al., (2001) Peripheral anti-A beta antibody alters CNS and plasma A beta clearance and decreases brain A beta burden in a mouse model of Alzheimer's disease. Proc Natl Acad Sci U S A 98:8850-8855; Deane, et al, (2003) RAGE mediates amyloid-beta peptide transport across the blood-brain barrier and accumulation in brain. Nat Med 9:907-913), To determine whether CD45 deficiency impacted relative Aβ abundance in cerebral and systemic compartments, brains and plasma from CD45-deficient and -sufficient PSAPP mice were probed using a biochemical approach. The total insoluble Aβ species (including Aβ₁₋₄₀ and Aβ₁₋₄₂) in PSAPP/CD45−/− and PSAPP/CD45 mouse brain homogenates at 4 and 8 months of age were probed by ELISA.

Separate extracts of extracellular and intracellular proteins were prepared from mouse brain homogenates as described above. Quantification of total Aβ species (including Aβ_{1-40, 42}) was performed according to published methods (Rezai-Zadeh, et al., (2005) Green tea epigallocatechin-3-gallate (EGCG) modulates amyloid precursor protein cleavage and reduces cerebral amyloidosis in Alzheimer transgenic mice. j Neurosci 25:8807-8814), Total soluble Aβ species in blood plasma and extracellular/intracellular Aβ in brain homogenates were detected at 1:4 and 1:20 dilutions, respectively. Detergent-insoluble total Aβ species were detected in brain by extracting pellets in 5 M guanidine HCl buffer, followed by a 1:20 dilution in lysis buffer. Aβ_{1-40, 42} was quantified in all samples using Aβ_{1-40, 42} ELISA kits (IBL-America, Inc., Minneapolis, Minn.) in accordance with the instructions of the manufacturer, except that standards included 0.25 M guanidine HCl buffer in some cases.

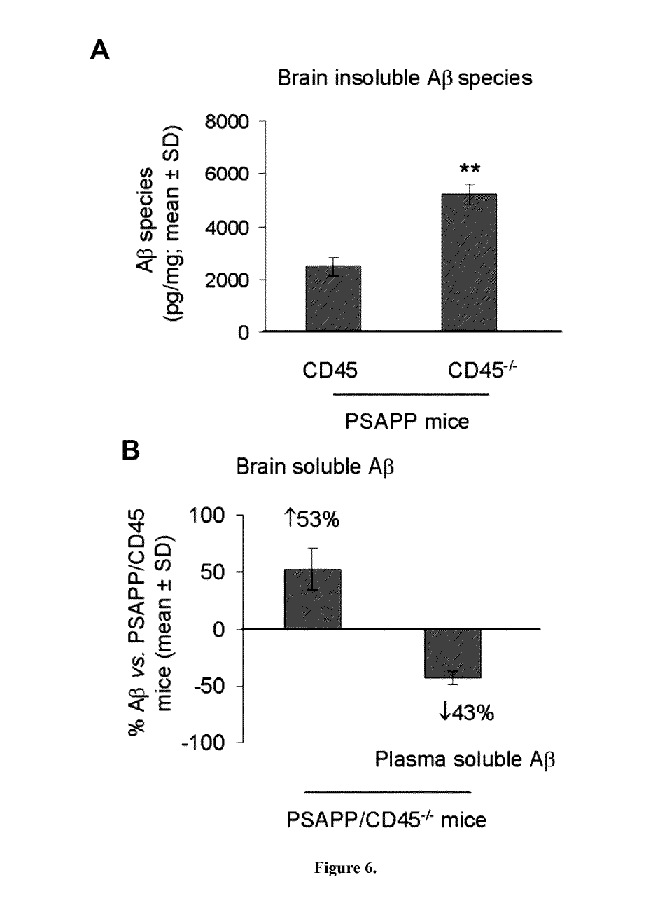

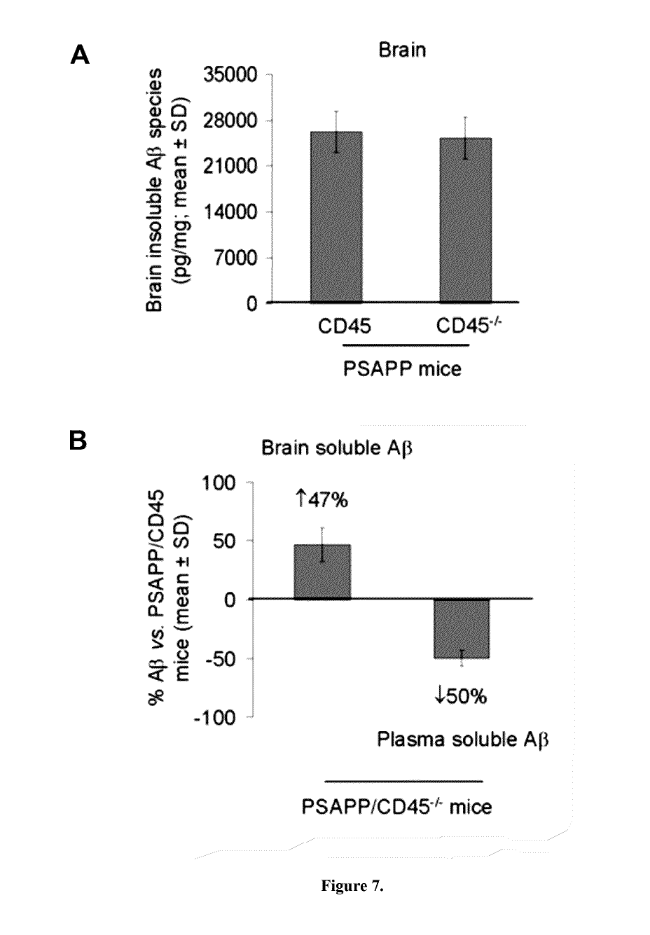

Analysis of 4-month-old mouse brains revealed significantly (**p<0.01) elevated abundance of Insoluble Aβ species in CD45-deficient versus-sufficient PSAPP mice, as seen in FIG. 6(A), although this difference was not evident in 8-month-old brains, as seen in FIG. 7(A). Correspondingly, cerebral detergent-soluble Aβ species were increased whereas plasma-soluble Aβ abundance was diminished by a similar magnitude at both 4 and 8 months of age in PSAPP/CD45−/− versus PSAPP/CD45 animals, as seen in FIGS. 6(B) and 7(B). Together, these results suggest that PSAPP/CD45−/− mice have Impaired brain-to-blood Aβ clearance.

### EXAMPLE 3

CD45 deficiency was analyzed to determine the inflammatory effect on microglia in PSAPP mice. Microglia are activated in close vicinity of β-amyloid plaques in AD patient brains and in transgenic mouse models of the disease (Benzing, et al., (1999) Evidence for glial-mediated inflammation in aged APP(SW) transgenic mice. Neurobiol Aging 20:581-589; Jimenez, et al., (2008) Inflammatory response in the hippocampus of PSIM146L/APP751SL mouse model of Alzheimer's disease: age-dependent switch in the microglial phenotype from alternative to classic. J Neurosci 28:11650-11661; Mandrekar-Colucci and Landreth, (2010) Microglia and inflammation in Alzheimer's disease. CNS Neurol Disord Drug Targets 9:156-167). Although it was once thought that microglial “activation” was a single phenotype, it is now known that multiple forms of functionally distinct reactive microglia, exist (Town, et al., (2005) The microglial “activation” continuum: from innate to adaptive responses. J Neuroinflammation 2:24; Colton, (2009) Heterogeneity of microglial activation in the innate immune response in the brain. J Neuroimmune Pharmacol 4:399-4.18; Colton and Wilcock; (2010) Assessing activation states in microglia. CNS Neurol Disord Drag Targets 9:174-191).

To determine whether CD45 deficiency impacted microglial phenotype in PSAPP mice, brain sections from PSAPP/CD45 and PSAPP/CD45−/− mice were stained with antibodies directed against the activated microglial markers Ibal, CD11b, or CD40 (Tan, et al., (1999) Microglial activation resulting from CD40-CD40L interaction, after beta-amyloid stimulation. Science 286:2352-2355; Townsend, et al., (2005) CD40 signaling regulates innate and adaptive activation of microglia in response to amyloid beta-peptide. Eur J Immunol 35:901-910; Ahmed, et al, (2007) Actin-binding proteins coronin-1a and IBA-1 are effective microglial markers for immunohistochemistry. J Histochem Cytochem 55:687-700), in combination with Aβ antibody 4G8 and DAPI as a nuclear counterstain. Briefly, brain sections were stained with rat antimouse CD11b (1:1000; AbD Serotec, Kidlington, UK), fluorescein, isothiocyanate-(FITC)-conjugated hamster anti-mouse CD40 (1:100; BD Biosciences Pharmingen), rabbit anti-mouse ionized calcium binding adaptor molecule 1 (Ibal) (1:1.000; Wako Pure Chemicals Chemical Industries, Ltd. Osaka, JP), hamster anti-mouse CD11c (1:50; Thermo Fisher Scientific Inc., Rockford, Ill. rat anti-mouse chemokine receptor Ccr2 (1:100; Novus Biologicals, LLC, Littleton, Colo.), mouse anti-human Aβ (clones 4G8 or 6E10; 1:500; Covance. Inc.). or mouse anti-NeuN (1:3000; Millipore Corp.). Brain sections were incubated with species-specific Alexa Fluor 488- and 594-conjugated secondary antibodies (Invitrogen) for 1 b at room temperature, followed by staining with the VECTASTAIN Elite ABC kit (Vector Laboratories, Inc.) coupled with 3,3′-diaminobenzidine substrate. Sections were analyzed in independent channels with an Olympus FY1000 laser scanning confocal microscope equipped with Fluoview SV1000 imaging software.

Because microglia activate in response to Aβ deposits and 4-month-old PSAPP/CD45^{-/-} mice had elevated β-amyloid plaque Load versus controls, as seen in FIG. 1. To avoid this confounder, the analysis focused on 8-month-old cohort with minimal or no differences on insoluble Aβ abundance, as seen in FIG. 7(A). Ibal-positive microglia were generally found in close spatial proximity to cortical Aβ plaque centers in PSAPP/CD45 mice (data not shown), whereas PSAPP/CD45^{-/-} animals displayed a more random and diffuse pattern of parenchymal Ibal reactivity. Furthermore, the distance between each microglial cell to the center of the nearest Congo red-positive Aβ plaque was measured in brain sections from 8-month-old PSAPP/CD45^{-/-} versus PSAPP/CD45 mice. Briefly, slides were rinsed in distilled water and dehydrated in 95% ethanol. After dehydration, slides were mounted with mounting medium and visualized in bright field. Congo red staining was performed as described previously (Shankar, et al., 2008, Amyloid-beta protein dimers isolated directly from Alzheimer's brains impair synaptic plasticity and memory, Nat Med 14:837- 842).

At the quantitative level, Ibal-positive cells were significantly (**p<0.01) farther away from Aβ deposits when comparing PSAPP/CD45−/− with PSAPP/CD45 mice. The diffuse nature of Jbal -positive microglia in PSAPP/CD45^{-/-} mice seems consistent, with a. “runaway” pro-inflammatory state that is poorly directed toward amyloid plaques in these mice. Similar results were observed in PSAPP/CD45^{-/-} mice at 4 months of age, despite imbalance in cerebral amyloidosis in this cohort (data not shown).

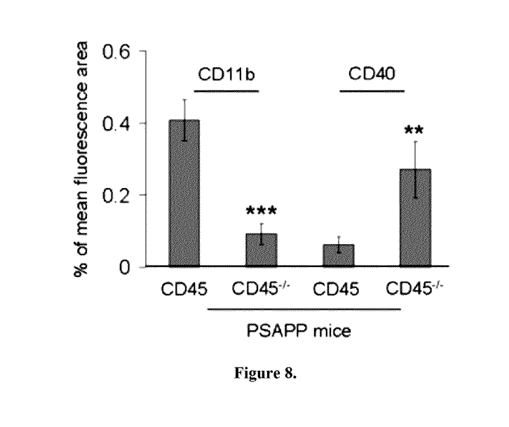

Immunological co-stimulation via the CD40 pathway was shown to enable microglial inflammatory responses after Aβ peptide stimulation and also reduce Aβ clearance responses by these cells (Tan, et al., (1999) Microglial activation resulting from CD40-CD40L interaction after beta-amyloid stimulation. Science 286:2352-2355; Tan, et al., (2002) Role of CD40 ligand in. amyloidosis in. transgenic Alzheimer's mice. Nat Neurosci 5:1288-1293; Townsend, et al, (2005) CD40 signaling regulates innate and adaptive activation of microglia in response to amyloid beta-peptide. Eur J Immunol 35:901-910). Consistent with a pro-inflammatory but anti-Aβ phagocytic microglial phenotype, Aβ plaque-associated microglia in PSAPP/CD45^{-/-} brains loose CD11b signal but gain expression of CD40 (data not shown). Quantification of these results revealed a statistically significant (***p<0.005) reduction in CD11b but significantly (**p<0.01) increased CD40 signal in PSAPP/CD45^{-/-} versus PSAPP/CD45 mouse brains as seen in FIG. 8. To further assess brain inflammation, the microglial pro-inflammatory cytokines TNF-γ and IL-1β were measured in brain homogenates from PSAPP/CD45 and PSAPP/CD45^{-/-} mice at 4 and 8 months of age.

Total proteins for TNF-α and interleukin-1β (IL-1β) were extracted from mouse brain homogenates as described above. Supernatants were collected and diluted them at 1:4 in lysis buffer for detection of TNF-α (R & D Systems, Inc., Minneapolis, Minn.) or IL-1β (eBioscience, Inc., San Diego, Calif.), in accordance with the instructions of the manufacturer. Total protein concentrations were determined for each brain sample before quantification of cytokines by ELISA to allow for sample normalization.

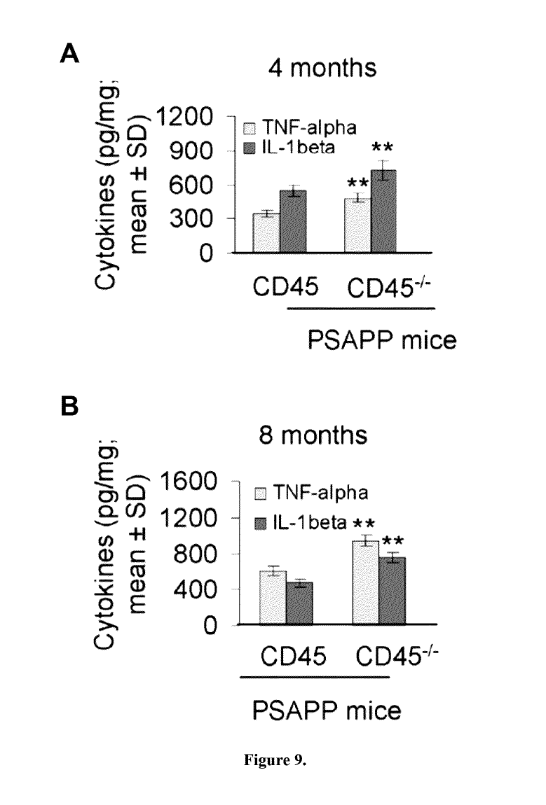

Consistent with histological observations, data revealed significantly (**p<0.0.1) increased levels of both cytokines in CD45-deficient versus-sufficient PSAPP mice at both ages, as seen in FIGS. 9(A) and (B). When, taken, together with the above Aβ plaque microglial localization findings, these results suggest that CD45 deficiency promotes an inflammatory microglial phenotype that, is inefficient at restricting cerebral amyloidosis and promotes buildup of neurotoxic Aβ oligomers.

To better characterize whether CD45 deficiency affected microglia or blood-borne monocytes/macrophages (or both), an immunofluorescence approach based on morphologic and immunophenptypic criteria was used to critically examine brain sections for any evidence of hematogenously derived immune cells (El Khoury, et al., (2007) Ccr2 deficiency impairs microglial accumulation and accelerates progression of Alzheimer-like disease. Nat Med 13:432-438; Town, et al., (2008) Blocking TGF-beta-Smad2/3 innate immune signaling mitigates Alzheimer-like pathology, Nat Med 14:681-687). This methodology was chosen over irradiation/bone marrow chimeras, because the latter have become controversial Specifically, the act of irradiating mice artificially sensitizes the CNS to large-scale infiltration and engraftment of the adoptively transferred peripheral macrophages (Ahmed, et al, 2007. Actin-binding proteins coronin-1a and IBA-1 are effective microglial markers for immunohistochemistry, J Histochem Cytochem 55:687-700; Mildner, et. al., 2007, Microglia in the adult brain arise from Ly-6ChiCCR2+ monocytes only under defined host conditions, Nat Neurosci 10; 1544-1553).

Brain sections were stained with rat antimouse CD11b (1:1000; AhD Serotec, Kidlington, UK), fluorescein isothiocyanate (FITC)-conjugated hamster anti-mouse CD40 (1:100; BD Biosciences Pharmigen), rabbit anti-mouse ionized calcium binding adaptor molecule 1 (Ibal) (1:1000; Wako Pure Chemicals Chemical Industries, Ltd. Osaka, JP), hamster anti-mouse CD11c (1:50; Thermo Fisher Scientific Inc., Rockford, Ill.), rat anti-mouse chemokine receptor Ccr2 (1:100; Novus Biologicals, LLC, Littleton, Colo.), mouse anti-human Aβ (clones 4G8 or 6E10; 1:500; Covance, Inc.), or mouse anti-NeuN (1:3000; Millipore Corp.). Brain sections were incubated with species-specific Alexa Fluor 488- and 594-conjugated secondary antibodies (Invitrogen) for 1 h at room temperature, followed by staining with die VECTASTAIN Elite ABC kit (Vector Laboratories, Inc.) coupled with 3,3′-diaminobenzidine substrate. Sections were analyzed in independent channels with an Olympus FV1000 laser scanning confocal microscope equipped with Fluoview SV1000 imaging software.

Despite careful determination of CD3, CD4, CD45 (data not shown), Ibal, CD11c, and Ccr2 expression and inclusion of an experimental autoimmune encephalomyelitis-positive control, no blood-derived immune cells were detected in any of the four mouse groups analyzed, (data not shown).

CD45^{-/-} microglia were tested for Aβ₁₋₄₂ phagocytosis capacity. Although there has been much recent debate about whether microglia are efficient Aβ phagocytes (Grathwohl, et al., (2009) formation and maintenance of Alzheimer's disease beta-amyloid plaques in the absence of microglia. Nat Neurosci 12:1361-1363), microglial Aβ phagocytosis has nonetheless been suggested to occur to a limited extent in the AD brain (Familian, et al., (2007) Minocycline does not affect amyloid beta phagocytosis by human microglial cells. Neurosci Lett 416:87-91), and it was recently shown that peripherally derived mononuclear phagocytes can clear oligomeric Aβ species (Town, et al., (2008) Blocking TGF-beta-Smad2/3 innate immune signaling mitigates Alzheimer-like pathology. Nat Med. 14:681-687), in vivo results suggested that CD45 deficiency promoted a proinflammatory yet anti-Aβ phagocytic microglial phenotype (Town, et al., (2005) The microglial “activation” continuum: from, innate to adaptive responses. J Neuroinflammation 2:24).

To determine whether CD45 agonism could produce the converse in vitro, CD45-deficient and -sufficient microglia were prepared from neonates as described previously (Zhu, et al., (2008) CD45RB is a novel molecular therapeutic target to inhibit Abeta peptide-induced microglial MAPK activation. PLoS One 3:e2135) and challenged with agonistic CD45 antibody or isotype matched control IgG in the presence of aged FITC-Aβ₁₋₄₂. Briefly, primary mouse microglia were seeded at 1×10⁵ cells per well (n=6 for each condition) in 24-well tissue culture plates containing 0.5 ml of complete RPMI 1640 medium for fluorometric analysis of Aβ. These cells were treated for 2 h with “aged” Aβ₁₋₄₂ [500 nM; preaggregated for 24 h at 37° C. in complete medium as described by Chung et al (Chung, et al, (1999) Uptake, degradation, and release of fibrillar and soluble forms of Alzheimer's amyloid beta-peptide by microglial cells. J Biol Chem 274:32301-32308) and conjugated with FITC (FITC-Aβ₁₋₄₂) (Bachem Americas)]. Microglial cells were then co-treated with agonistic CD45 antibody or isotype control IgG (2.5 μg/ml.) for 2 h in the presence of FITC-Aβ_{1-42.} Cells were then rinsed three times in Aβ-free complete medium, and the medium was exchanged with fresh Aβ-free complete medium for 10 min to allow for removal of non-incorporated Aβ and to promote concentration of Aβ into phagosomes. Extracellular (in cell culture media) and cell-associated (in cell lysates) FITC-Aβ were quantified using an MSF SpectraMax spectrophotometer (Molecular Devices, Corp., Sunnyvale, Calif.) with an emission wavelength of 538 nm and an excitation wavelength of 485 nm. A standard curve from 0 to 600 nM FITC-Aβ was generated for each plate. Total cellular proteins were quantified by BCA. Protein. Assay. The mean fluorescence values for each sample at 37° C. and 4° C. at the 2 h time point were determined by fluorometric analysis. Relative fold change values were calculated as follows: mean fluorescence value for each sample at 37° C./mean fluorescence value for each sample at 4° C. Considering nonspecific adherence of Aβ to the plastic surface of culture plates, an additional control without cells was performed in parallel for each experiment above. An incubation time of <4 h did not change the amount of Aβ peptide detected in the supernatant, consistent with a previous report (Mitrasinovic and Murphy Jr (2002) Accelerated phagocytosis of amyloid-beta by mouse and human microglia overexpressing the macrophage colony-stimulating factor receptor, J Biol Chem 277:29889-29896). To determine whether cell death influenced Aβ uptake in the various treatment groups, lactate dehydrogenase release assays were performed, but did not detect significant (p<0.05) cell death over the 3 h experimental timeframe in any of the treatment groups (data, not shown).

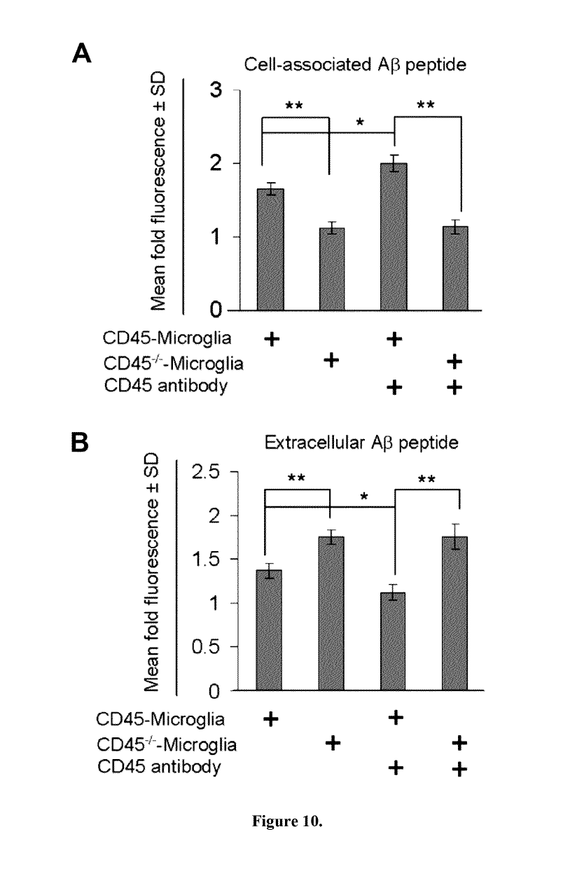

As shown in FIG. 10(A), ablation of CD45 significantly (**p<0.0.1) diminished microglial phagocytosis of FITC-Aβ₁₋₄₂, and addition of agonistic CD45 antibody significantly (*p<0.05) elevated this effect m CD45-sufficient cells (but had no effect on CD45-deficient control cells). Microglia treated with a non-relevant isotype matched IgG control antibody did not differ from untreated cells (data not shown). To validate this result, primary microglia were treated as described above and then imaged them by confocal microscopy.

“Aged” FITC-Aβ₁₋₄₂ was prepared according to methods described above. Microglial cells were cultured at 1×105 cells per well in 24-well tissue culture plates with glass inserts. These cells were treated for 2 h with aged FITC-Aβ₁₋₄₂. Separate groups of microglial cells were incubated, in parallel at 4° C. (control). After treatment, cells were washed five times with ice-cold PBS to remove non-incorporated FITC-Aβ₁₋₄₂ and fixed for 10 min at 4° C. in 4% PFA, followed by three rinses in PBS. Finally, sections were mounted with fluorescence mounting media containing DAPI (ProLong Gold; Life Tech.) and viewed with a Leica SP5 confocal microscope (Leica Microsystems GmbH, Wetzlar, Del.). Excitation wavelengths of 488 nm (to reveal FITC-Aβ₁₋₄₂ and 405 nm (for DAPI counterstained nuclei) were used. Images were captured and analyzed using LAS AF software version 1.6.0 (Leica Microsystems GmbH). Normarski optic (differential interference contrast) images were captured in wide field to accompany each confocal image.

Data revealed FITC-Aβ₁₋₄₂ peptide within the cytoplasm of CD45-sufficient primary microglial cells, whereas the peptide remained on the surface of CD45-deficient cells (data not shown). Interestingly, unlike the more ramified appearance of wild-type cells that typically indicates a “resting” state, CD45-deficient microglia had a unique morphology denoted by an ovoid cytoplasm, and relatively few cytoplasmic processes compared with wild-type cells (data not shown). This morphological phenotype of CD45-deficient microglia occurred in concert with strikingly increased expression of CD40, as seen in FIG. 8, a key costimulatory protein required for proinflammatory innate immune activation of antigen presenting cells. Furthermore, ovoid CD45-deficient microglia were unable to take up fluorescently tagged Aβ peptide in vitro. Thus, without being bound to any specific theory, it appears reasonable to conclude that CD45 deficiency leads to a functional switch in microglial phenotype characterized by morphologic and immunophenotypic changes consistent with an activated, proinflammatory state that is incompatible with Aβ clearance. Although this particular microglial phenotype seems to be deleterious in the context of AD, it is important to note that not all forms of microglial activation are detrimental; this is underscored by findings from. Aβ “immunotherapy” approaches, in which microglia could be stimulated to phagocytose and clear Aβ deposits decorated with Aβ-specific antibodies (Schenk, et al., (1999) Immunization with amyloid-beta attenuates Alzheimer-disease-like pathology in the PDAPP mouse. Nature 400; 173-177; Bard, et al, (2000) Peripherally administered antibodies against amyloid beta-peptide enter the central nervous system and reduce pathology in. a mouse model of Alzheimer disease. Nat Med 6:9.16-919).

Increased neuronal intracellular Aβ in aged PSAPP/CD45^{-/-} mice

Aβ can exist in both secreted and intracellular pools within the brain (Watson, et al., (2005) Physicochemical characteristics of soluble oligomeric Abeta and their pathologic role in Alzheimer's disease. Neurol Res 27:869-881). APP is normally metabolized to Aβ via an endocytosis-dependent, pH-sensitive pathway (Shop, et al., (1992) Production of the Alzheimer amyloid beta protein by normal proteolytic processing. Science 258:126-129; Koo and Squazzo, (1994) Evidence that production and release of amyloid beta-protein involves the endocytic pathway. J Biol Chem 269:17386-17389). and intracellular Aβ has been found in degenerating neurons in the AD brain (Probst, et al., (199) Deposition of beta/ A4 protein along neuronal plasma membranes in diffuse senile plaques. Acta Neuropathol 83:21-29). If CD45-deficient microglia were unable to effectively clear cerebral Aβ, then one might expect intracellular buildup of the peptide. To evaluate this, intracellular Aβ was analyzed in 4- and 8-month-old PSAPP/CD45 and PSAPP/CD45^{-/-} brain sections by immunostaining. Brain sections were stained with mouse anti-human Aβ (clones 6E10; 1:500; Covance, Inc.). Brain sections were incubated with species-specific Alexa Fluor 488- and 594-conjugated secondary antibodies (Invitrogen) for 1 h at room temperature, followed by staining with the VECTASTAIN Elite ABC kit (Vector Laboratories, Inc.) coupled with 3,3′-diaminobenzidine substrate. Sections were analyzed in independent channels with an Olympus FV1000 laser scanning confocal microscope equipped with Fluoview SV1000 imaging software.

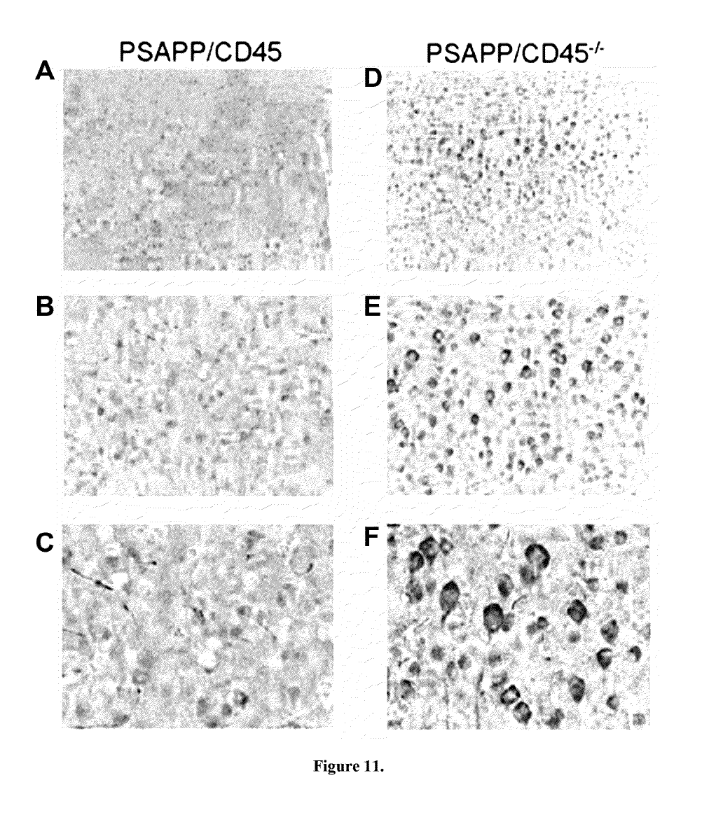

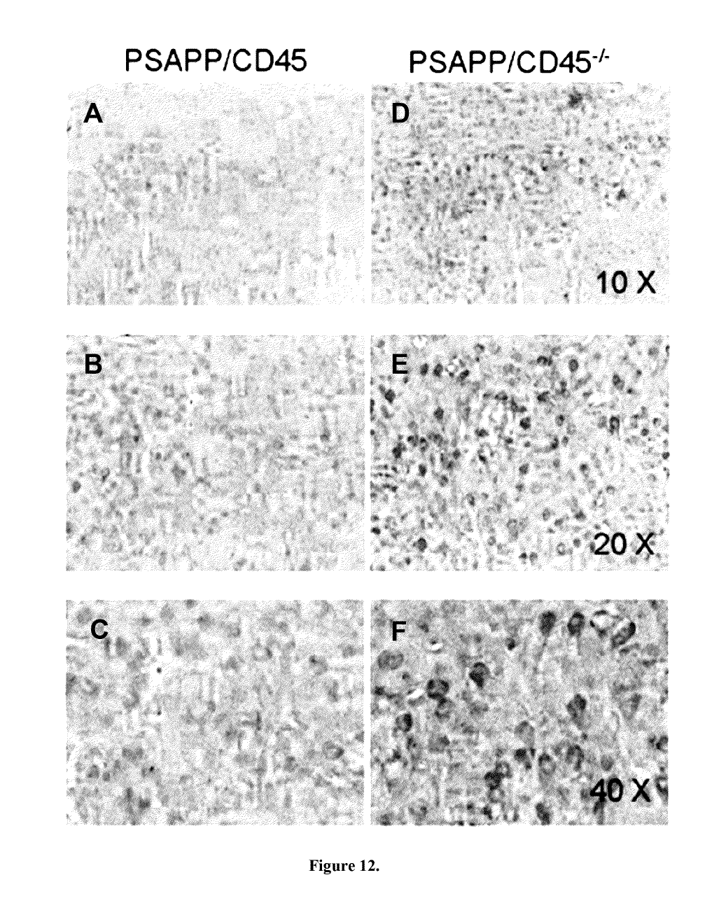

Regardless of age, CD45-deficient mouse brains showed a marked increase in intraneuronal 6E10 reactivity, as seen in FIGS. 11(A) through 12(I). To confirm the Aβ identity of these signals. Western immunoblot was performed by 6E.10 antibody.

Following the sample preparation as described above, an aliquot corresponding to 40 μg of total protein was electrophoretically separated using 10% Tris-SDS gels or 10-20% Tris-tricine gels (Bio-Rad Laboratories, Hercules, Calif.) and transferred to polyvinylidene fluoride membranes (Bio-Rad Laboratories). As a positive control, AP oligomers were prepared from synthetic human Aβ₁₋₄₂ according to published methods (Walsh, et al., (2000) The oligomerization of amyloid beta-protein begins intracellularly in cells derived from human brain. Biochemistry 39:10831-10839; Lesné, et al., (2006) A specific amyloid-beta protein assembly in the brain impairs memory. Nature 440:352-357). Membranes were blocked for 1 h at room temperature in Tris buffered saline (TBS) (containing 0.1% Tween 20 with 5% nonfat dry milk) and were then incubated with primary antibody for mouse monoclonal 6E10 (1:2000; Covance, Inc., Princeton, N.J.). Afterward, membranes were immunoblotted with anti-mouse (1:2000; Cell Signaling Technology, Inc.) IgG secondary antibody conjugated with horseradish peroxidase. Proteins were detected with Super Signal West Femto Maximum Sensitivity Substrate (Pierce) and BIOMAX-MR Film (Thermo Fisher Scientific).

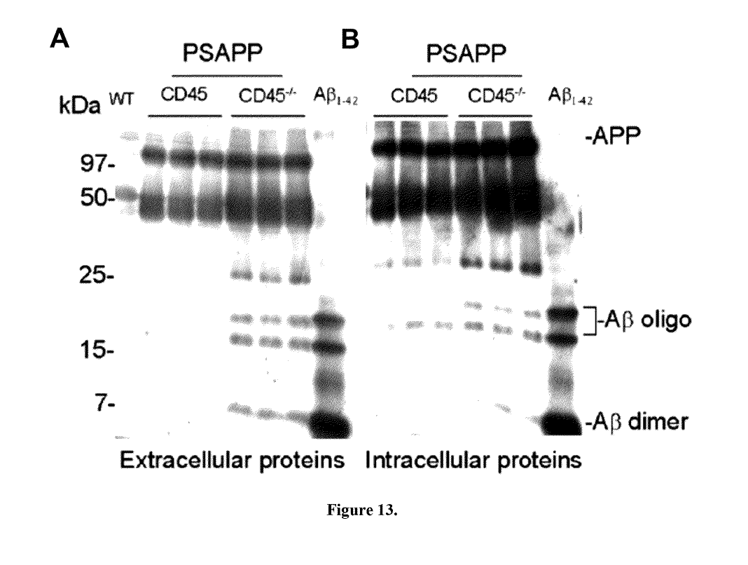

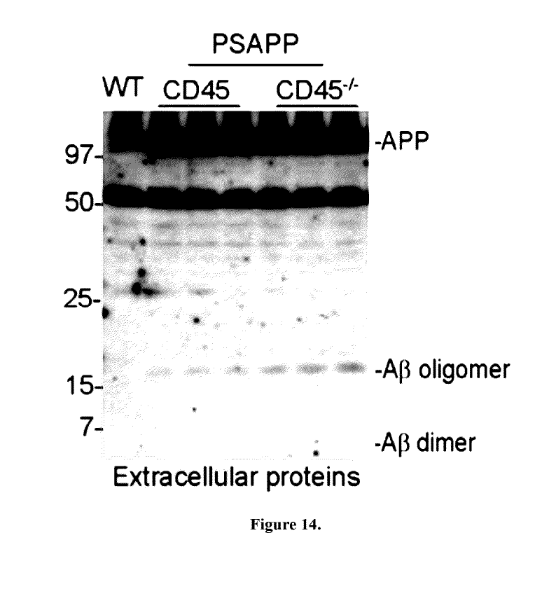

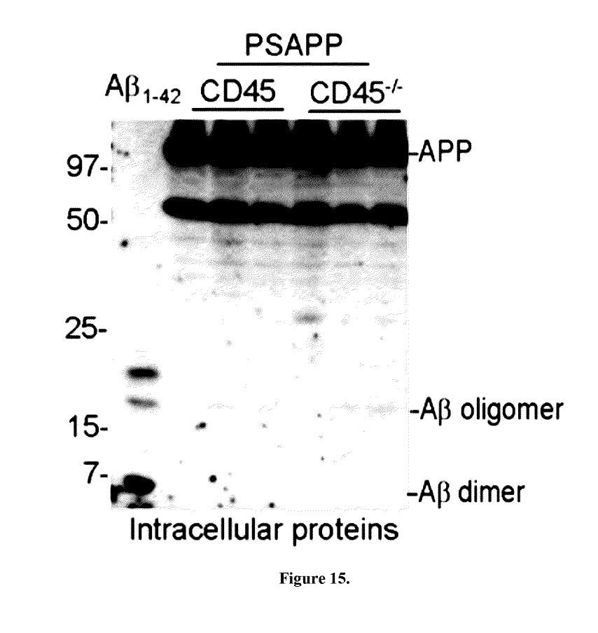

It was observed that extracellular and intracellular dimeric and oligomeric Aβ species were increased in PSAPP/CD45^{-/-} versus PSAPP/CD45 mice at 8 months, as seen in FIGS. 13(A) and (B), and a similar pattern of results was also likely the case at 4 months of age, as seen in FIGS. 14 and 15, although the investigation occurred at the detection, limit, for the assay at this earlier age. Additionally, extracellular and intracellular soluble Aβ were quantified from PSAPP/CD45 and PSAPP/CD45^{-/-} mouse brains by ELISA.

Separate extracts of extracellular and intracellular proteins were prepared from mouse brain homogenates as described above. Quantification of total Aβ species (including Aβ_{1-40, 42}) was performed according to published methods (Rezai-Zadeh, et al., (2005) Green tea epigallocatechin-3-gallate (EGCG) modulates amyloid precursor protein cleavage and reduces cerebral amyloidosis in Alzheimer transgenic mice. J Neurosci 25:8807-8814). Total soluble Aβ species in blood plasma and extracellular/intracellular Aβ in brain homogenates were detected at 1:4 and 1:20 dilutions, respectively. Detergent-insoluble total Aβ species were detected in brain by extracting pellets in 5 M guanidine HCl buffer, followed by a 1:20 dilution in lysis buffer. Aβ_{1-40, 42} was quantified in all samples using Aβ_{1-40, 42} ELISA kits (IBL-America, Inc., Minneapolis, Minn.) in accordance with the instructions of the manufacturer, except that standards included 0.25 M guanidine HCl buffer in some cases.

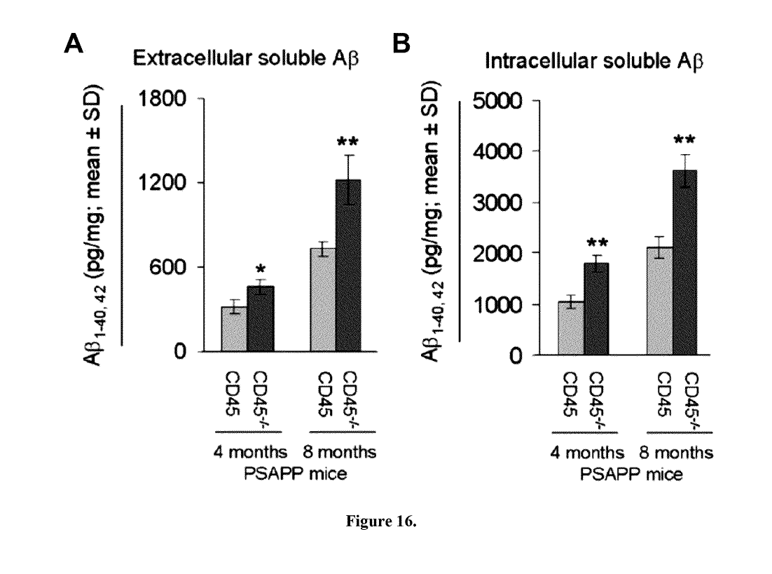

Data revealed significantly (*p<0.05; **p<0.01) increased abundance of total soluble intracellular Aβ species in PSAPP/CD45^{-/-} versus PSAPP/CD45 mice at both ages and in both fractions, as seen in FIGS. 16(A) and (B).

### EXAMPLE 5

An important hallmark of AD is loss of neurons, resulting in significant atrophy of the cerebral cortex and certain subcortical regions, including the temporal lobe, parietal lobe, parts of the frontal cortex, and the cingulate gyrus (Wenk, (2003) Neuropathologic changes in Alzheimer's disease. J Qui Psychiatry 64 [Suppl 9]:7-10). As discussed supra, intraneuronal Aβ is increased in PSAPP/CD45^{-/-} mice. As such, this form of Aβ pathology was investigated for co-occurrence of neuronal loss in PSAPP/CD45−/− mice.

Forty μm free-floating serial brain, sections were stained in plastic multiwell carriers with nylon net bottoms using NeuN or Nissl antibody. Briefly, free-floating frozen sections were mounted on. slides and air dried before overnight incubation with a 1:1. solution of alcohol and chloroform. Afterward, sections were rehydrated through a graded series of alcohols and. distilled, water and stained with 0.1% cresyl violet solution for 5-1.0 min. Slides were then rinsed in distilled water and dehydrated in 95% ethanol After dehydration, slides were mounted with mounting medium and visualized in bright field. Congo red staining was performed as described previously (Shankar, et al, (2008) Amyloid-beta protein dimers isolated directly from Alzheimer's brains impair synaptic plasticity and memory. Nat Med 14:837-842). To prepare for unbiased stereologic estimation of neuronal counts, an initial tissue section was randomly selected at one anatomic border of the brain region to be examined. Thereafter, every sixth section throughout the anatomic region of interest was used for each counting series. NeuN-positive cells were examined with a Nikon Eclipse 600 microscope and quantified using Stereo Investigator software, version 6 (MicroBrightField, Inc., Williston, Vt.). Cells were counted in the entorhinal. cortex using the optical fractionator method of unbiased stereologic cell counting (West, et al., (1991) Unbiased stereological estimation of the total number of neurons in the subdivisions of the rat hippocampus using the optical fractionator. Anat Rec 231:482-497). The sampling method was optimized to count at least 250 cells per animal with error coefficients of <0.07. Each counting frame (50×50 μm) was placed at an intersection of the lines forming a virtual grid (200×200 μm), which was randomly generated and placed by the software within the outlined structure.

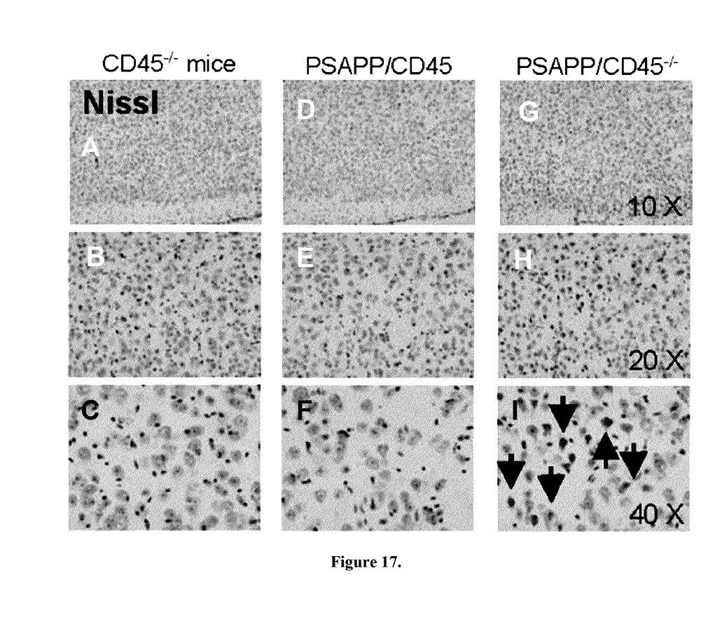

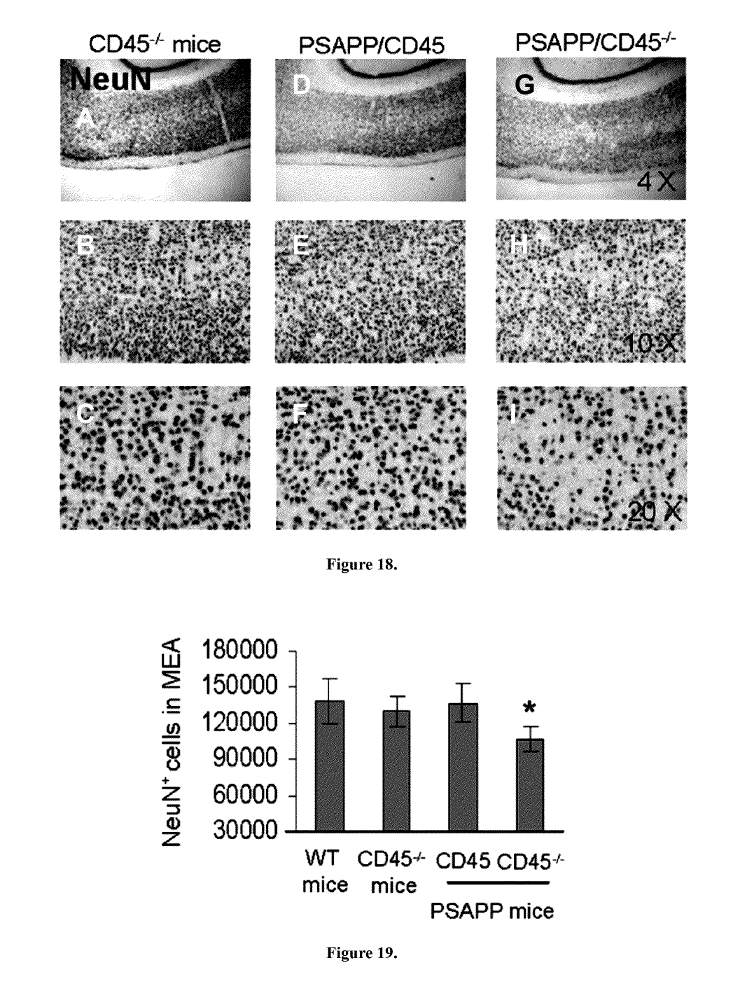

Stereological analysis was performed for Nissl and NeuN-positive cells (data not shown), Nissl staining revealed neuronal dysmorphology suggestive of neuronal degeneration, as seen in FIGS. 17(A)-(I). Furthermore, NenN immonohistochemistry disclosed a more rarefied pattern of neurons in PSAPP/CD45^{-/-} mice, seen in FIG. 18(A)-(I), and stereological analysis demonstrated significantly (*p<0.05) decreased NeuN-positive cells in the medial entorhinal cortex, seen in FIG. 19 of PSAPP/CD45^{-/-} versus PSAPP/CD45 or control CD45^{-/-} mice at 8 months of age, but this was not yet evident in 4-month-old animals (data not shown).

To validate these results, brain homogenates were prepared from each group of mice at 8 months of age. Western blot analysis was performed on PSAPP/CD45^{-/-} versus PSAPP/CD45 or control CD45^{-/-} mice. Following the sample preparation as described above, an aliquot corresponding to 40 μg of total protein was electrophoretically separated using 10% Tris-SDS gels or 10-20% Tris-tricine gels (Bio-Rad Laboratories, Hercules, Calif.) and transferred to polyvinylidene fluoride membranes (Bio-Rad Laboratories), As a positive control, Aβ oligomers were prepared from synthetic human Aβ₁₋₄₂ according to published methods (Walsh, et al, (2000) The oligomerization of amyloid beta-protein begins intracellalarly in cells derived from human brain. Biochemistry 39:10831-10839; Lesné, et al., (2006) A specific amyloid-beta protein assembly in the brain impairs memory. Nature 440:352-357). Membranes were blocked for 1 h at room temperature in Tris buffered saline (TBS) (containing 0.1% Tween 20 with 5% nonfat dry milk) and were then incubated with primary antibodies including mouse monoclonal neuronal-specific nuclear protein (NeuN) (1:1000; Millipore Corporation, Billerica, Mass.)., rabbit polyclonal Bcl-xL or Bax (1:1000; Cell Signaling Technology, Inc., Danvers, Mass.), mouse monoclonal 6E10 (1:2000; Covance, Inc., Princeton, N.J.), or mouse monoclonal β-actin (1:4000; Sigma-Aldrich Co. LLC., St. Louis, Mo.). Afterward, membranes were immunoblotted with anti-mouse (1:2000; Cell Signaling Technology, Inc.) or anti-rabbit (1:10,000; Thermo Fisher Scientific Inc., Rockford, Ill.) IgG secondary antibodies conjugated with horseradish peroxidase. Proteins were detected with Super Signal. West Femto Maximum Sensitivity Substrate (Pierce) and BIOMAX-MR Film (Thermo Fisher Scientific).

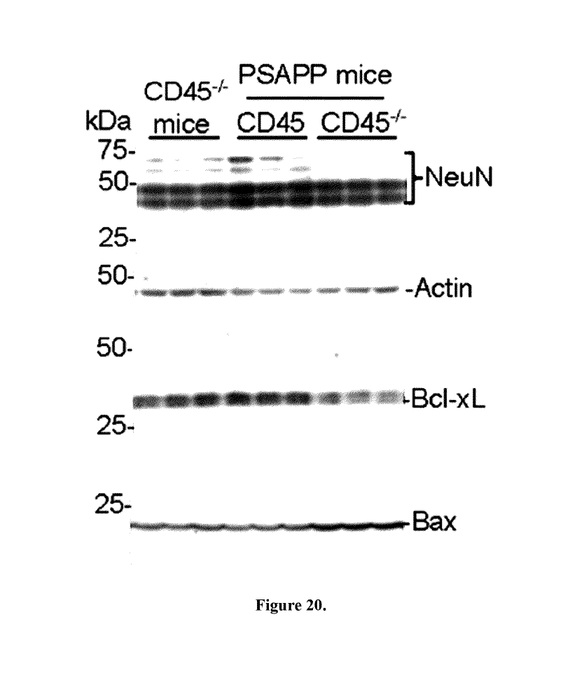

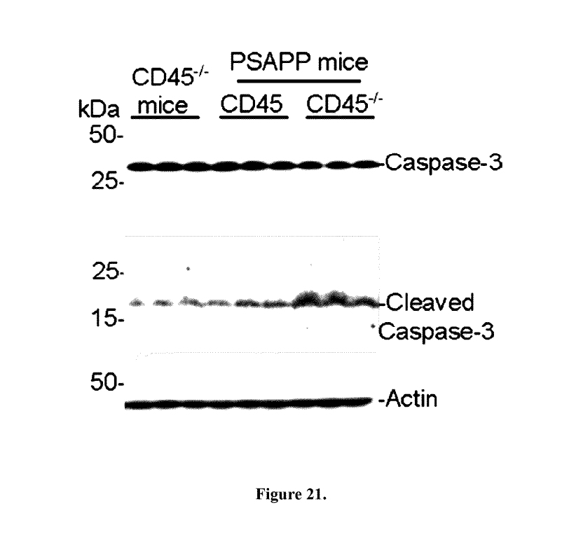

Western blot analysis revealed decreased levels of NeuN relative to actin in PSAPP/CD45^{-/-} versus PSAPP/CD45 or control CD45^{-/-} mice, as seen in FIG. 20. A similar pattern of results was noted when considering expression ratio of the neuronal anti-apoptotic regulator Bcl-xL (Parsadanian, et al., (1998) Bcl-xL is an antiapoptotic regulator for postnatal CNS neurons. J Neurosci 18:1009-1019) to the proapoptotic protein Bax, as seen in FIG. 20. Furthermore, another index of apoptosis, cleaved caspase-3, was overrepresented in PSAPP/CD45^{-/-} mice compared with the other two mouse groups, whereas unprocessed caspase-3 did not differ between groups, as seen in FIG. 21. When taken together, these results demonstrate neuronal loss in PSAPP/CD45^{-/-} mice, likely as a result of apoptosis.

Mitochondrial dysfunction in PSAPP/CD45^{-/-} mice

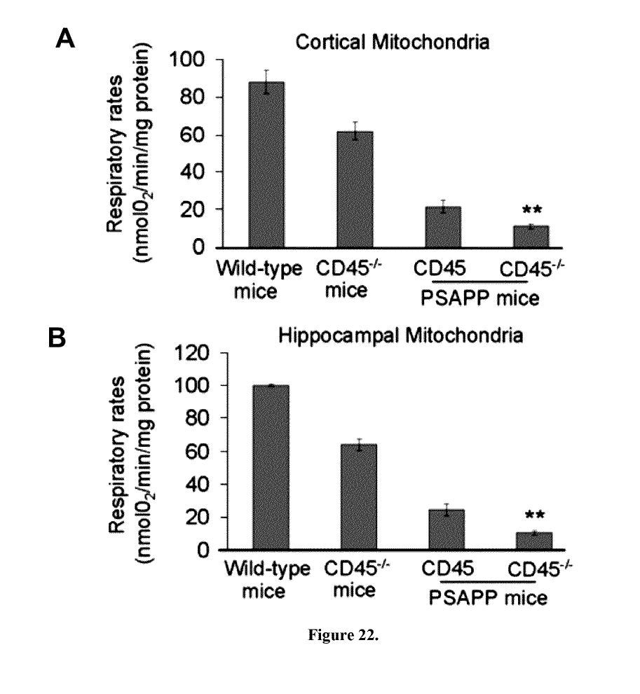

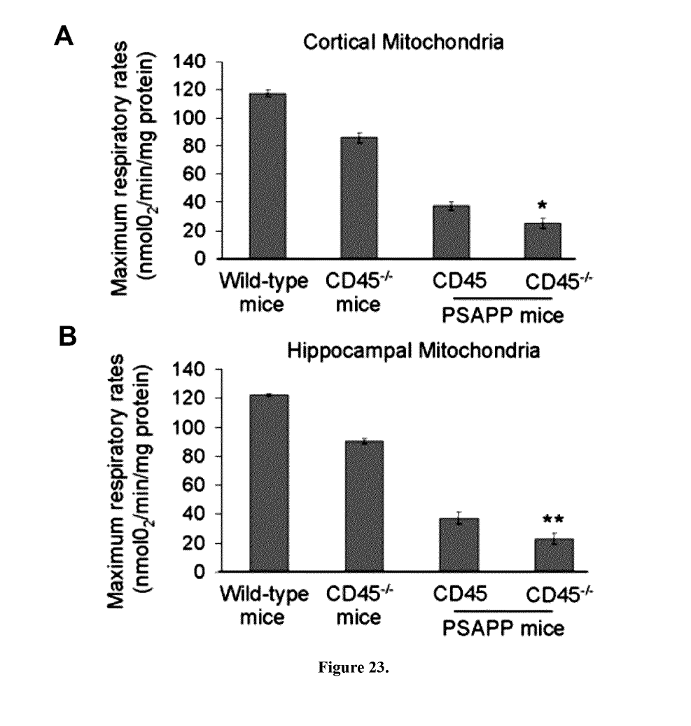

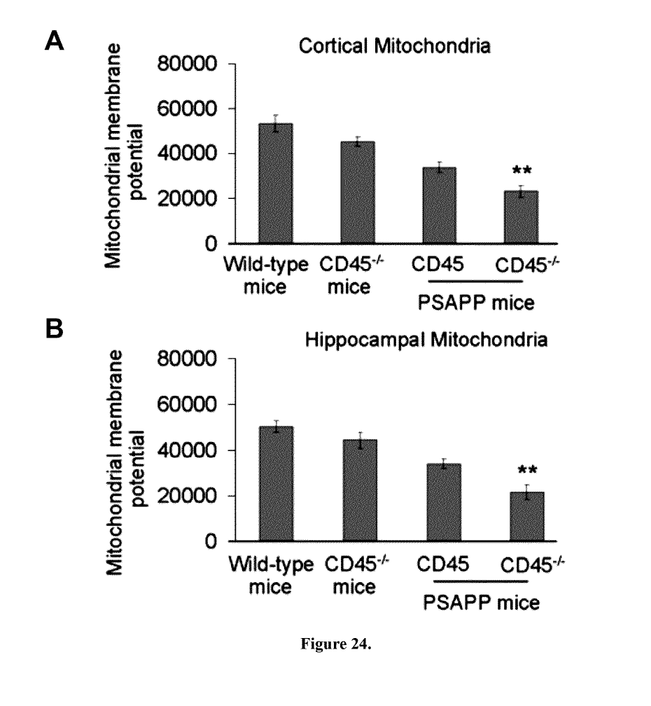

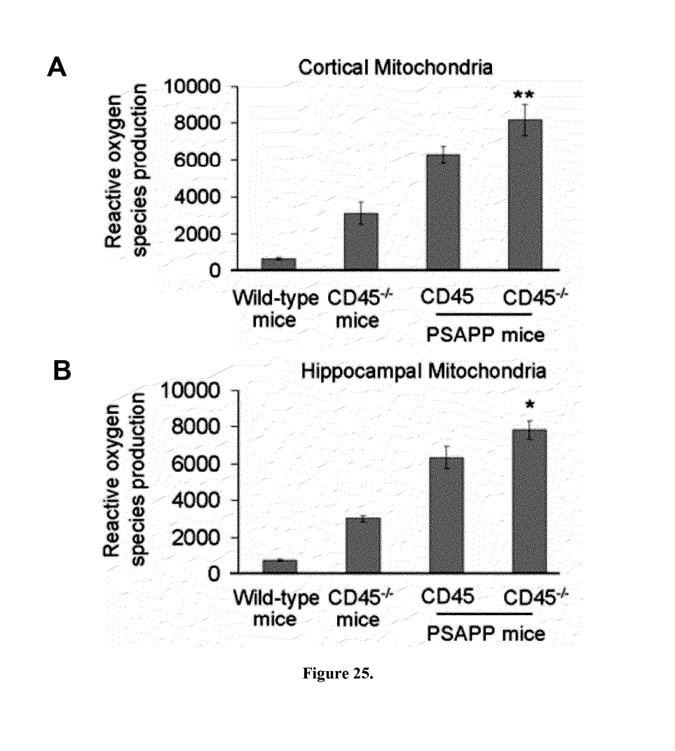

The brain is highly dependent on aerobic metabolism, and mitochondria are responsible for cellular respiration. To investigate whether neuronal, loss in PSAPP/CD45^{-/-} mice was accompanied by loss of mitochondrial function, mitochondria were isolated from cortical regions (including frontal, entorhinal, and cingulated cortices) and hippocampi of 8-month-old wild-type, CD45^{-/-}, PSAPP/CD45, and PSAPP/CD45^{-/-} mice. Respiratory rates were then enumerated for each brain region in all mouse groups. Significantly reduced basal (state II) respiration was observed, as seen in (*p<0.05; **<0.01), as seen in FIGS. 22(A) and (B) and attenuated maximum respiratory rate, seen in FIGS. 23(A) and (B), in PSAPP/CD45^{-/-} mice versus the three other groups for all brain regions examined. Furthermore, mitochondrial membrane potential, seen in FIGS. 24(A) and (B), and reactive oxygen species abundance, seen in FIGS. 25(A) and (B), were significantly (*p<0.05; **p<0.0l) reduced in PSAPP/CD45^{-/-} compared with wild-type, CD45^{-/-} or PSAPP/CD45 mice for mitochondria isolated from either cortical or hippocampal brain regions. These results indicate that PSAPP/CD45^{-/-} mice exhibit mitochondrial dysfunction, which dovetails with shift from antiapoptotic to proapoptotic proteins and neuronal loss in these animals.

There has been considerable recent debate surrounding the relationship between microglia and AD-like pathology. Although microglia in brains of healthy elderly individuals are uniformly distributed, these cells appear in tight temporal and spatial proximity to amyloid plaques in brains of AD patients and in transgenic mouse models of the disease (MeGeer, et al., (1987) Reactive microglia in patients with senile dementia of the Alzheimer type are positive for the histocompatibility glycoprotein HLA-DR, Neurosci Lett 79:195-200; Brazing, et al., (1999) Evidence for glial-mediated inflammation in aged APP(SW)transgenic mice. Neurobiol Aging 20:581-589; Akiyama, et al., (2000) Inflammation and Alzheimer's disease, Neurobiol Aging 21:383-421; Heneka and O'Banion, (2007) Inflammatory processes in Alzheimer's disease. J Neuroimmunol 184:69 -91). These pathological observations have prompted the conclusion that microglia are etiological participants in AD, although this remains controversial (Grathwohl, et al., (2009) Formation and maintenance of Alzheimer's disease beta-amyloid plaques in the absence of microglia. Nat Neurosci 12:1361-1363). In support of this notion, studies that impair microglial or mononuclear phagocyte functions by (1) treatment with nonsteroidal anti-inflammatory drugs (Lim, et al., (2000) Ibuprofen suppresses plaque pathology and inflammation in a mouse model for Alzheimer's disease, J Neurosci 20:5709 -5714, Lim, et al, (2001) The curry spice curcumin reduces oxidative damage and amyloid pathology in an Alzheimer transgenic mouse. J Neurosci 21:8370-8377), (2) interrupting CD40-CD40L interaction (Tan, et al., (1999) Microglial activation resulting from CD40-CD40L interaction after beta-amyloid stimulation. Science 286:2352-2355; Tan, et al. (2002) Role of CD40 ligand in amyloidosis in transgenic Alzheimer's mice. Nat Neurosci 5:1288-1293), or (3) genetically ablating transforming growth factor-α (TGF-α) receptor signaling (Town, et al, (2008) Blocking TGF-beta-Smad2/3 innate immune signaling mitigates Alzheimer-like pathology. Nat Med 14:681-687) mitigate AD-like pathology in transgenic AD mice. Additionally, immunotherapy approaches that rely on Aβ-specific antibodies to stimulate microglial clearance of Aβ deposits resolve AD-like pathology in mouse models (Schenk, et ah, (1999) Immunization with amyloid-beta attenuates Alzheimer-disease-like pathology in the PDAPP mouse. Nature 400:1.73-177; Bard, et al., (2000) Peripherally administered antibodies against amyloid beta-peptide enter the central nervous system and reduce pathology in a mouse model of Alzheimer disease. Nat Med 6:916-919). However, deficiency of the Ccr2 chemokine receptor reduces microglial recruitment to brains of AD model mice and causes accumulation of cerebral amyloid plaques (El Khoury, et al., (2007) Ccr2 deficiency impairs microglial accumulation and accelerates progression of Alzheimer-like disease. Nat. Med .13:432- 438), whereas genetic ablation of the Cx3crl fractalkine receptor impairs microglial migration to neurons “marked for death” and prevents neuronal loss in 3xTg-AD mice (Fuhrmann, et al, (2010) Microglial Cx3crl knockout prevents neuron loss in a mouse model of Alzheimer's disease, Nat Neurosci 13:411- 413). A parsimonious conclusion that arises from these results is that multiple forms of microglial activation exist some being deleterious and others, beneficial (Town, et al., (2005) The microglial “activation” continuum: from innate to adaptive responses, J Neuroinflammation 2:24; Wyss-Coray, (2006) Inflammation in Alzheimer disease: driving force, bystander or beneficial response? Nat Med 12:1005-1015).

CD45 is the most abundant membrane-bound protein tyrosine phosphatase and functions to dampen overly exuberant immune responses (Justement (1997) The role of CD45 in signal transduction, Adv Immunol 66:1-65). Furthermore, microglial CD45 abundance is increased in brains of AD patients and in mouse models of the disease (Masliah, et al., (1991) Immunoreactivity of CD45, a protein phosphotyrosine phosphatase, in Alzheimer's disease. Acta Neuropathol 83:12-20; Licastro, et al., (1998) Increased levels of alpha-1-antichymotrypsin in brains of patients with Alzheimer's disease correlate with activated astrocytes and are affected by APOE 4 genotype. J Neoroimmunol 88:105-110). Although multiple variants of CD45 are generated by alternate mRNA splicing, the CD45RB isoform is most highly expressed by microglia (Townsend, et al, (2004) CD45 isoform RB as a molecular target to oppose lipopolysaccharideinduced microglial activation in mice. Neuroses Lett 362:26-30). Microglial CD45 functions- to inhibit nitric oxide and TNF-α production induced by Aβ) peptides (Tan, et al., (2000) CD45 opposes p-amyloid peptide-induced microglial activation via inhibition of p44/42 mitogen-activated protein kinase. J Neurosci 20:7587-7594), CD40L, or bacterial endotoxin by dephosphorylating Src-family kinases and thereby inactivating p44/42 and p38 MAPKs (Zhu, et al, (2008) CD45RB is a novel molecular therapeutic target to inhibit Abeta peptide-induced microglial MAPK activation. PLoS One 3:e2135). Without being bound to any specific theory, in vitro CD45RB appears to act on microglia as a molecular switch to turn phagocytosis “off” and damaging proinflammatory response “on” in the presence of exogenous Aβ (Zhu, et al, (2008) CD45RB is a novel molecular therapeutic target to inhibit Abeta peptide-induced microglial. MAPK activation. PLoS One 3:e2135).

To explore the functional consequences of CD45 deficiency in vivo, transgenic mice overproducing Aβ were crossed with animals deficient in CD45, PSAPP/CD45−/− mice manifest accelerated cerebral amyloidosis, characterized by elevated abundance of β-amyloid plaques and both intracellular and extracellular pools of soluble, oligomeric, and insoluble Aβ. Although soluble intraneuronal forms of Aβ are produced under physiologic conditions, a tight balance exists between peptide production and clearance (Shoji et al., (1992) Production of the Alzheimer amyloid beta protein by normal proteolytic processing. Science 258:126-129; Koo and Squazzo, (1994) Evidence that production and release of amyloid beta-protein involves the endocytic pathway. J Biol Chem 269:17386-17389); yet, abnormally high amounts of intracellular Aβ are present in degenerating neurons in brains of AD and Down's syndrome patients (Allsop, et al., (1989) Early senile plaques in Down's syndrome brains show a close relationship with cell bodies of neurons. Neuropathol Appl Neurobiol 15:531-542; Probst, et al., (1991) Deposition of beta/A4 protein along neuronal plasma membranes in diffuse senile plaques. Acta Neuropathol 83:21-29), in monkey and rodent models of Aβ deposition (Martin, et al, (1994) Synaptic pathology and glial responses to neuronal injury precede the formation of senile plaques and amyloid deposits in the aging cerebral cortex. Am J Pathol 145:1358-1381), and in human immunodeficiency virus patients with dementia (Green, et al., (2005) Brain deposition of beta-amyloid, is a common pathologic feature in HIV positive patients. AIDS 19:407-411). Intracellular Aβ is produced in the endoplasmic reticulum and Golgi complex in neuronal cells (Wertkin. et al., (1993) Humanneurons derived from a teratocarcinoma cell line express solely the 695-amino acid amyloid precursor protein and produce intracellular beta-amyloid or A4 peptides. Proc Natl Acad Sci USA 90:9513-9517; Wild-Bode, et al, (1997) Intracellular generation and accumulation of amyloid betapeptide terminating at amino acid 42. J Biol Chem 272:16085-16088; Xu, et al, (1997) Generation of Alzheimer beta-amyloid protein in the trans-Golgi network in the apparent absence of vesicle formation. Proc Natl Acad Sci U S A 94:3748-3752), and Aβ immunoreactivity within lysosomes of degenerating neurons has been found in both aging macaques (Martin, et al, (1994) Synaptic pathology and glial responses to neuronal injury precede the formation of senile plaques and amyloid deposits in the aging cerebral cortex. Am J Pathol 145:1358-1381) and in Aβ-infused rats (Frautschy, et al, (1996) Rodent models of Alzheimer's disease: rat A beta infusion approaches to amyloid deposits, Neurobiol Aging 17:311-321). The results from PSAPP/CD45^{-/-} mice lend support to the idea that intraneuronal Aβ accumulation precedes neuronal loss, as recently suggested by another group (Fuhrmann, et. ah, (2010) Microglial Cx3crl knockout prevents-neuron loss in a mouse model of Alzheimer's disease, Nat Neurosci 13:411-413).

Recent studies indicate that soluble oligomeric Aβ species may function as the agents of neurotoxicity. Administration of Aβ oligomers directly isolated from AD patient, cerebral cortices to normal rats impaired long-term potentiation, enhanced long-term depression, and reduced dendritic spine density. Furthermore, these deleterious effects were specifically attributable to Aβ dimers (Shankar, et al, (2008) Amyloid-beta protein dimers isolated directly from Alzheimer's brains impair synaptic plasticity and memory. Nat Med 1.4:837-842), and it is likely that Aβ-directed. immunotherapy works by clearing oligomeric species of Aβ (Klynbin, et al., (2005) Amyloid beta protein immunotherapy neutralizes Abeta oligomers that disrupt synaptic plasticity in vivo. Nat Med 11:556-561). However, previous studies are unclear as to whether mononuclear phagocytes, including microglia, impact steady-state Aβ oligomer abundance. Blockade of TGF-α-Smad 2/3 signaling was previously shown to promote uptake and clearance of Aβ oligomers by cells of mononuclear phagocyte origin (Town, et at, (2008) Blocking TGF-beta-Smad2/3 innate immune signaling mitigates Alzheimer-like pathology. Nat Med 14:681-687), and the present work demonstrates that CD45 deficiency drives accumulation of cerebral Aβ dimers and oligomers in a transgenic mouse model of AD, Results presented here place microglia on the Aβ oligomer clearance pathway and suggest that ablating CD45 and thereby inhibiting this clearance machinery causes buildup of neurotoxic Aβ oligomers and neuropathology downstream of the amyloid cascade (Hardy and Allsop, (1991) Amyloid deposition as the central event in the aetiology of Alzheimer's disease. Trends Pharmacol Sci 12:383-388).

A number of studies have shown that the BBB is responsible for elimination of human Aβ from the brain into the blood (Shibata, et. al., (2000) Clearance of Alzheimer's amyloid-ss(1-40) peptide from brain by LDL receptor related protein-1 at the blood-brain barrier. J Clin Invest 106:1489-1499; Shiiki, et al., (2004) Brain insulin impairs amyloid-β₁₋₄₀ clearance from the brain. J Neurosci 24:9632-9637; Terasaki and Ohtsuki, (2005) Brain-to-blood transporters for endogenous substrates and xenobiotics at the blood-brain barrier: an overview of biology and methodology. NeuroRx 2:63-72). Some have even harnessed this peripheral sink to clear cerebral amyloid by passive Aβ immunotherapy (DeMattos, et al, (2001) Peripheral anti-A beta antibody alters CNS and plasma A beta clearance and decreases brain A beta burden in a mouse model of Alzheimer's disease. Proc Natl Acad Sei USA 98:8850-8855; DeMattos, et al., (2002) Brain to plasma amyloid-beta efflux; a measure of brain amyloid burden in a mouse model of Alzheimer's disease. Science 295:2264-2267). Aβ transport across the BBB is bidirectional because, when exogenous human Aβ₁₋₄₀ is systemically injected, it is transported into the brain (Martel, et al., (1997) isoform-specific effects of apolipoproteins E2, E3, and E4 on cerebral capillary sequestration and blood-brain barrier transport, of circulating Alzheimer's amyloid beta. J Neorochem 69; 1995-2004; Wengenack, et al., (2000) Quantitative histological analysis of amyloid deposition in Alzheimer's double transgenic mouse brain. Neuroscience 101:939 -944: Deane, et al, (2003) RAGE mediates amyloid-beta peptide transport across the blood-brain barrier and accumulation in brain. Nat. Med 9:907-913). As such, Aβ BBB transport homeostasis is likely an important factor governing accumulation of cerebral Aβ (Ito, et al., (2006) Functional characterization of the brain-to-blood efflux clearance of human amyloid-beta peptide (1-40) across the rat blood-brain barrier. Neurosci Res 56:246 -252). In addition to elevated, cerebral amyloidosis, PSAPP/CD45^{-/-} mice also demonstrate decreased plasma-soluble Aβ abundance, likely reflective of diminished brain-to-blood Aβ efflux. Given the exquisite microglial specificity of CD45 expression in the brain, it is unlikely that CD45 deficiency directly impacts Aβ clearance at the BBB. A more likely possibility consistent with these observations is that failure in microglial Aβ clearance in PSAPP/CD45^{-/-} mice overloads brain-to-blood Aβ efflux machinery; leading to increased cerebral amyloid and reduced circulating Aβ.

In addition to accelerated cerebral amyloidosis, TNF-α and IL-1β abundance [which are neurotoxic at high levels (Meda, et al., (1995) Activation of microglial cells by beta-amyloid protein and interferon-gamma. Nature 374:647-650; Barger and Harmon, (1997) Microglial activation by Alzheimer amyloid precursor protein and modulation by apolipoprotein E. Nature 388:878-881; Tan, et al, (1999) Microglial activation resulting from CD40-CD40L interaction after beta-amyloid stimulation. Science 286:2352-2355)], mitochondrial dysfunction, and neuronal loss were found to increase in PSAPP/CD45^{-/-} mice. Association among these three observations leads to a model wherein CD45 deficiency endorses a proinflammatory but anti-Aβ phagocytic form of microglial activation. Because of failed microglial clearance of cerebral Aβ and overexpression of neurotoxic cytokines, downstream events in this model would include dysfunctional mitochondrial respiration and ultimately neuronal loss. However, it is unclear whether mitochondrial dysfunction brought on by loss of CD45 is a cause or consequence of neurotoxicity. In this regard, oxidative phosphorylation—and in particular cytochrome c oxidase activity—is deficient in AD patient, brains (Cottrell, et al., (200.1) Mitochondrial, enzyme-deficient hippocampal neurons and choroidal cells in AD. Neurology 57:260-264; Fukui, et al, (2007) Cytochrome c oxidase deficiency in neurons decreases both oxidative stress and amyloid formation in a mouse model of Alzheimer's disease. Proc Natl Acad Sci USA 104:14163-14168). However, falloff in cytochrome c oxidase activity is likely related to global decline in numbers of mitochondria as a result of neurotoxicity. A number of factors might contribute to the observed reduction in oxidative phosphorylation activity in AD, including failed mitochondrial transport through axonal and dendritic processes, compromised regulatory feedback mechanisms responsible for individual complex subunit synthesis, and impaired complex assembly (Mancuso et al., (2008) Mitochondria, mitochondrial DNA and Alzheimer's disease. What comes first? Curr Alzheimer Res 5:457- 468).

It is well established that CD45 has multiple splice variants (chiefly, -RA, -KB, -RC, and -RO), that are variously expressed by different immune cells. CD45 isoforms may functionally differ, and this explains why gross CD45 deficiency can lead to both hypo- and hyper-responsive immunological defects. In the case of microglia, 90% of CD45 was previously found to be accounted for by the CD45RB isoform (Townsend, et al, (2004) CD45 isoform RB as a molecular target to oppose lipopolysaccharideinduced microglial activation in mice. Neurosci. Lett 362:26-30). Furthermore, although agonistic antibodies directed against CD45RA or CD45RC isoforms had minimal effects on lipopolysaccharideinduced microglial activation, antibody mediated stimulation of CD45RB resulted in almost complete shutdown of lipopolysaccharide-induced TNF-α release in cultured microglia (Townsend, et al., (2004) CD45 isoform RB as a molecular target to oppose lipopolysaccharideinduced microglial activation in mice. Neurosci Lett. 362:26-30; Zhu, et al., (2008) CD45RB is a novel molecular therapeutic target to inhibit Abeta peptide-induced microglial MAPK. activation. PLoS One 3:e2135). Finally, stimulation of CD45RB specifically enhanced Aβ uptake that was dependent on inhibition of the p44/42 MAPK signaling cascade (Zhu, et al., (2008) CD45RB is a novel molecular therapeutic target to inhibit Abeta peptide-induced microglial MAPK activation. PLoS One 3:e2l35).

Genetic loss of CD45 was shown herein to (1) accelerate cerebral amyloidosis, (2) cause brain accumulation of soluble oligomeric Aβ species and reduction in plasma-soluble Aβ, (3) promote proinflammatory and anti-Aβ phagocytic microglial, activation, and (4) lead to mitochondrial dysfunction and neuronal loss in PSAPP/CD45^{-/-} mice.

In the preceding specification, all documents, acts, or information disclosed do not constitute an admission that the document, act, or information of any combination thereof was publicly available, known to the public, part of the general, knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a mouse model of Alzheimer's disease, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A mouse model of amyloid disease comprising a mouse having:
a haplotype derived from a PSAPP mouse:
a haplotype derived from an CD45 deficient mouse having a 100% deficiency;
wherein the mouse possesses a two-fold elevated level of amyloid proteins compared to wild type; and
wherein the mouse model exhibits impaired amyloid clearance.

2. The mouse model of claim L wherein the haplotype derived from a PSAPP mouse is derived from a double transgenic “Swedish” APPK595N/M596L strain mouse and a PSIE9 B6C3-Tg 85Dbo/J strain mouse.

3. The mouse model of claim 1. wherein the haplotype derived from, a CD45 deficient mouse is derived, from a B6.129-PtprctmlHolm/J strain mouse.

4. The mouse model of claim 1, wherein the mouse is only female.

5. The mouse model of claim 1, wherein the elevated levels of amyloid proteins are compared to wild-type mice, and wherein the amyloid proteins are dimeric Aβ, oligomeric Aβ or a combination, thereof.

6. The mouse model of claim 5, wherein the elevated levels of amyloid proteins are total soluble intracellular Aβ species.

7. The mouse model of claim 6, wherein the elevated levels of amyloid proteins are cerebral detergent-soluble Aβ, and further comprising a decreased level of plasma-soluble Aβ.

8. The mouse model of claim 1, wherein the mouse further has mitochondrial dysfunction.

9. The mouse model of claim 8, wherein the .mitochondrial dysfunction is activation of NADPH oxidase.

10. The mouse model of claim 1, wherein the amyloid disease is Alzheimer's disease.

11. A method of forming a mouse model of amyloid disease comprising:
obtaining a first filial parent having a genotype derived from a PSAPP mouse;
obtaining a second .filial parent having a genotype derived from a CD45 deficient mouse;
interbreeding the first filial parent with the second filial parent, to form first generational mouse model having a heterozygous PSAPP haplotype and a homozygous CD45-deficient haplotype; and
screening the interbred mouse for PSAPP and CD45 genotypes;
wherein the PSAPP mice were maintained as heterozygotes by crossing transgenic mice to wild-type B6C3FI/J mice.

12. The method of claim 11, wherein the screening is performed by PCR from genomic DNA or flow analysis of peripheral monocytes.

13. The method of claim 11, wherein the first filial parent overproduces Aβ.

14. The method of claim 11, wherein the mouse models are female.
